SEQUENCE LISTING (23 sequences)

SEQ ID NO: 1 - DNA, 3108 bp, Rattus sp.
gatcctgagtctcggtggattgaagtcgttgtccctgggggaggcaagagcttcagaaatcgcttacggtggaaaagatgcctgccttcaacagattgcttcccctagcttctctagtgctcatctactgggtcagagtctgcttccctgtgtgtgtggaagtgccctcggagacagaagcggtgcagggcaatcccatgaagctgaggtgcatctcctgcatgaagagggaggaggtggaggccaccactgtggtggagtggttctacaggcctgagggcggtaaagatttccttatatatgagtatcggaatggccaccaggaagtggagagccccttccaaggccgtctgcagtggaatgggagcaaagacctgcaggacgtatccatcactgtactcaatgtcactttgaatgactctggcctctacacatgcaatgtgtccagggagttcgaattcgaggcacacaggccttttgtgaagaccacgagactgatacctttgcgagtcactgaagaggcgggagaagacttcacctccgtggtctcggaaatcatgatgtacatcctcctggtcttcctcaccttgtggctgtttattgagatgatctattgctacagaaaggtctctaaggccgaagaggcagcacaggaaaatgcgtctgactaccttgctatcccttcagagaacaaggagaactctgtggtacctgtggaggaataatgtggtgtgacttgaggtgatctgagtgctgagggactggatatccccagttcagtgatgccagcaatatcaggaagtgccccaggtgtcccaacacatccatcttttctattcatcaaccaccaacccaatgtgagattttcacctgacttccgaactctatcagaactctacacatctttaccttgcctgaaccgaagagccaacatctatctctacacggactaaacctcactctgttcttgcttccaaccaagtaactcccaacttaactagagttgttccctatgttccaaatgatttagacaagtactggagagtagtattacctctgccctgactgtctgtgactgggtcattctccactgcagcaaaaggatggatataaatcggaagaaagccctgactagtttgtcttaaagccaaagcgtgccacgtacgtactttgattcattgaagtcagtttttcctgcttctcagagcgccagaaagcagcatgcccctaatgcttgcagggacatcatctgtgtgcactggaacgctttctggagctcagtgtttggaggctgtatccccataatcctgaagacctggagcaaacccagaacttccaggaagtcccaaggaaggatccaggacagtttcagtaggtctcgaaaatgatataacacactcctgatattggaaacatggatgagtgacctttctggattgaaactcctcagttcttcatgtctcagtgtctgtggatcagtattagtcctcgttttacaggaggaaactgagactcacacaaggctgaacaggacatttaggggattaaactgggccagagatgactttcctgccaccaacctcacactccctgggatgagaggtatttttgaggactctaacattcagcatgccatttgcccagcggaagctgactgccacagatctgaggaactggaaaccaggtaagaaaacacagacggcatgagatagacttcaggatttcacacaaagatttgtgaatctgaagcatcctccaggagagacggcacccgagggcaatatctctgtgatgaaaaatggttttagtctgaaatggacagtcaacagagagacaaagatgggcgtgtagcttctaaatacctcacctgtagatgtcacgtttgggtactggtgtttgtaaagtcccccacctctaccctcatccccacccaagagaacagggaaccagtgacaagatgtttagtcaatatctctttttaatgactgttctaaatgcaatattctttagcccctccagatagcaagggcagaaggccttatgatgtataaaaaggtaaaagtgagactccataggagaaatgtcctcacctctggggagatctgtaagaaagaccattctccacccctacctggctttgtatgaaaagctcacctttcactcccaacatttccttcttggtggatgccagacatgcgctttgggaaccccatcccaccctacctttatctagcttgtttatacatggtggcttatctggaatcacaatcgcatttccttataggagatcatgactataacctacaaggcacggcactcaccagccaattaaaatctttgccatctccatttcttcccataattccacttagaatcctggactagacatgtgtgactctggagcctttcctggtgtgctatggcttactaaagggcttagaagagttctcaagggctataatcatccagaaaccccattgtttgtcttggtgctgggtatttgcccggactgattctcaataatttactgaaggtcaaactaagctgagtctagttttccccttatccctgcctaacagctgtgaacttggttactcagttggtcctgagagcatcttgccctgttggcttggacggtcagccaggggttgttaccgagccccaatccctaagcgcttgctatctcaggctttactttcaatctgtttacacattattctttggtttctccaccctcggtcaaccgcacaccccagctatcttttgagtttctgggtttgctctagacatcgaaatagaattgcaagtagaaagggaactttccagagcaagattatgctgagaacgacaggggtgaggttaaaaggcgttagggctaaggtgatagcccagtgggtaaaatgcttgccacccaagcgtgagagtctcagtgtgatctccagaatccatgtgaacagacagacattgtggcacacattggtcatcccagagctggggaggcagagacaggaagatctgagcctcactaaccaggccacctggcctaacttacttacttggcaagctccaggagagtgagacgctgtctcaaaaaaaaaaaaaaaaaaa

SEQ ID NO: 2 - protein, 215 aa, Rattus sp.
MPAFNRLLPLASLVLIYWVRVCFPVCVEVPSETEAVQGNPMKLRCISCMKREEVEATTVVEWFYRPEGGKDFLIYEYRNGHQEVESPFQGRLQWNGSKDLQDVSITVLNVTLNDSGLYTCNVSREFEFEAHRPFVKTTRLIPLRVTEEAGEDFTSVVSEIMMYILLVFLTLWLFIEMIYCYRKVSKAEEAAQENASDYLAIPSENKENSVVPVEE

SEQ ID NO: 3 - protein, 24 aa, Rattus sp.
MPAFNRLLPLASLVLIYWVRVCFP

SEQ ID NO: 4 - protein, 191 aa, Rattus sp.
VCVEVPSETEAVQGNPMKLRCISCMKREEVEATTVVEWFYRPEGGKDFLIYEYRNGHQEVESPFQGRLQWNGSKDLQDVSITVLNVTLNDSGLYTCNVSREFEFEAHRPFVKTTRLIPLRVTEEAGEDFTSVVSEIMMYILLVFLTLWLFIEMIYCYRKVSKAEEAAQENASDYLAIPSENKENSVVPVEE

SEQ ID NO: 5 - protein, 111 aa, Rattus sp.
VCVEVPSETEAVQGNPMKLRCISCMKREEVEATTVVEWFYRPEGGKDFLIYEYRNGHQEVESPFQGRLQWNGSKDLQDVSITVLNVTLNDSGLYTCNVSREFEFEAHRPFV

SEQ ID NO: 6 - protein, 22 aa, Rattus sp.
KTTRLIPLRVTEEAGEDFTSVV

SEQ ID NO: 7 - protein, 58 aa, Rattus sp.
SEIMMYILLVFLTLWLFIEMIYCYRKVSKAEEAAQENASDYLAIPSENKENSVVPVEE

SEQ ID NO: 8 - protein, 4 aa, Rattus sp.
NGSK

SEQ ID NO: 9 - protein, 4 aa, Rattus sp.
NVTL

SEQ ID NO: 10 - protein, 4 aa, Rattus sp.
NDSG

SEQ ID NO: 11 - protein, 4 aa, Rattus sp.
NVSR

SEQ ID NO: 12 - protein, 4 aa, Rattus sp.
NASD

SEQ ID NO: 13 - protein, 4 aa, Rattus sp.
RKVS

SEQ ID NO: 14 - protein, 3 aa, Rattus sp.
TTR

SEQ ID NO: 15 - protein, 4 aa, Rattus sp.
SETE

SEQ ID NO: 16 - protein, 4 aa, Rattus sp.
TVVE

SEQ ID NO: 17 - protein, 4 aa, Rattus sp.
TLND

SEQ ID NO: 18 - protein, 4 aa, Rattus sp.
SKAE

SEQ ID NO: 19 - protein, 6 aa, Rattus sp.
GLYTCN

SEQ ID NO: 20 - protein, 218 aa, Lepus Sp.
MGRLLAFVVGAALVSSAWGGCVEVDSETEAVYGMTFKILCISCKRRSETTAETFTEWTFRQKGTEEFVKILRYENEVLQLEEDERFEGRVVWNGSRGTKDLQDLSIFITNVTYNHSGDYQCHVYRLLSFENYEHNTSVVKKIHLEVVDKANRDMASIVSEIMMYVLIVVLTIWLVAEMVYCYKKIAAATEAAAQENASEYLAITSESKENCTGVQVAE

SEQ ID NO: 21 - DNA, 645 bp, Rattus sp.
atgcctgccttcaacagattgcttcccctagcttctctagtgctcatctactgggtcagagtctgcttccctgtgtgtgtggaagtgccctcggagacagaagcggtgcagggcaatcccatgaagctgaggtgcatctcctgcatgaagagggaggaggtggaggccaccactgtggtggagtggttctacaggcctgagggcggtaaagatttccttatatatgagtatcggaatggccaccaggaagtggagagccccttccaaggccgtctgcagtggaatgggagcaaagacctgcaggacgtatccatcactgtactcaatgtcactttgaatgactctggcctctacacatgcaatgtgtccagggagttcgaattcgaggcacacaggccttttgtgaagaccacgagactgatacctttgcgagtcactgaagaggcgggagaagacttcacctccgtggtctcggaaatcatgatgtacatcctcctggtcttcctcaccttgtggctgtttattgagatgatctattgctacagaaaggtctctaaggccgaagaggcagcacaggaaaatgcgtctgactaccttgctatcccttcagagaacaaggagaactctgtggtacctgtggaggaa

SEQ ID NO: 22 - DNA, 2632 bp, Rattus sp.
gatcctgagtctcggtggattgaagtcgttgtccctgggggaggcaagagcttcagaaatcgcttacggtggaaaagatgcctgccttcaacagattgcttcccctagcttctctagtgctcatctactgggtcagagtctgcttccctgtgtgtgtggaagtgccctcggagacagaagcggtgcagggcaatcccatgaagctgaggtgcatctcctgcatgaagagggaggaggtggaggccaccactgtggtggagtggttctacaggcctgagggcggtaaagatttccttatatatgagtatcggaatggccaccaggaagtggagagccccttccaaggccgtctgcagtggaatgggagcaaagacctgcaggacgtatccatcactgtactcaatgtcactttgaatgactctggcctctacacatgcaatgtgtccagggagttcgaattcgaggcacacaggccttttgtgaagaccacgagactgatacctttgcgagtcactgaagaggcgggagaagacttcacctccgtggtctcggaaatcatgatgtacatcctcctggtcttcctcaccttgtggctgtttattgagatgatctattgctacagaaaggtctctaaggccgaagaggcagcacaggaaaatgcgtctgactaccttgctatcccttcagagaacaaggagaactctgtggtacctgtggaggaataatgtggtgtgacttgaggtgatctgagtgctgagggactggatatccccagttcagtgatgccagcaatatcaggaagtgccccaggtgtcccaacacatccatcttttctattcatcaaccaccaacccaatgtgagattttcacctgacttccgaactctatcagaactctacacatctttaccttgcctgaaccgaagagccaacatctatctctacacggactaaacctcactctgttcttgcttccaaccaagtaactcccaacttaactagagttgttccctatgttccaaatgatttagacaagtactggagagtagtattacctctgccctgactgtctgtgactgggtcattctccactgcagcaaaaggatggatataaatcggaagaaagccctgactagtttgtcttaaagccaaagcgtgccacgtacgtactttgattcattgaagtcagtttttcctgcttctcagagcgccagaaagcagcatgcccctaatgcttgcagggacatcatctgtgtgcactggaacgctttctggagctcagtgtttggaggctgtatccccataatcctgaagacctggagcaaacccagaacttccaggaagtcccaaggaaggatccaggacagtttcagtaggtctcgaaaatgatataacacactcctgatattggaaacatggatgagtgacctttctggattgaaactcctcagttcttcatgtctcagtgtctgtggatcagtattagtcctcgttttacaggaggaaactgagactcacacaaggctgaacaggacatttaggggattaaactgggccagagatgactttcctgccaccaacctcacactccctgggatgagaggtatttttgaggactctaacattcagcatgccatttgcccagcggaagctgactgccacagatctgaggaactggaaaccaggtaagaaaacacagacggcatgagatagacttcaggatttcacacaaagatttgtgaatctgaagcatcctccaggagagacggcacccgagggcaatatctctgtgatgaaaaatggttttagtctgaaatggacagtcaacagagagacaaagatgggcgtgtagcttctaaatacctcacctgtagatgtcacgtttgggtactggtgtttgtaaagtcccccacctctaccctcatccccacccaagagaacagggaaccagtgacaagatgtttagtcaatatctctttttaatgactgttctaaatgcaatattctttagcccctccagatagcaagggcagaaggccttatgatgtataaaaaggtaaaagtgagactccataggagaaatgtcctcacctctggggagatctgtaagaaagaccattctccacccctacctggctttgtatgaaaagctcacctttcactcccaacatttccttcttggtggatgccagacatgcgctttgggaaccccatcccaccctacctttatctagcttgtttatacatggtggcttatctggaatcacaatcgcatttccttataggagatcatgactataacctacaaggcacggcactcaccagccaattaaaatctttgccatctccatttcttcccataattccacttagaatcctggactagacatgtgtgactctggagcctttcctggtgtgctatggcttactaaagggcttagaagagttctcaagggctataatcatccagaaaccccattgtttgtcttggtgctgggtatttgcccggactgattctcaataatttactgaaggtcaaactaagctgagtctagttttccccttatccctgcctaacagctgtgaacttggttactcagttggtcctgagagcatcttgccctgttggcttg

SEQ ID NO: 23 - DNA, 657 bp, Lepus Sp.
atggggaggctgctggctttcgtggtcggcgccgcgctggtgtcctcggcctgggggggctgcgtggaggtggactcggagaccgaagccgtgtacgggatgaccttcaaaattctgtgcatctcctgcaagcgccgcagcgagaccacggccgagaccttcacggagtggaccttccgccagaagggcactgaggagttcgtcaagatcctgcgctatgagaacgaggtgctgcagctggaggaagacgagcgctttgagggccgcgtggtgtggaacggcagccggggcaccaaggacctgcaggacctgtccatcttcatcaccaatgtcacctacaaccactcgggcgactaccagtgccatgtctaccgcctgctctccttcgaaaactacgagcacaacaccagcgtcgtcaagaagatccacctggaggtggtggacaaggccaacagagacatggcatccatcgtgtcggagatcatgatgtacgtgctcatcgtggtgttgaccatctggctcgtggcggagatggtgtactgctacaagaagatcgcggccgccacggaggcagcggcgcaggagaacgcctcggaatacttggccatcacctcagaaagcaaagaaaattgcacgggcgtccaggtggctgaatag

(19) United States
(12) Patent Application Publication
(10) Pub. No.: US 2002/0160453 A1
(43) Pub. Date: Oct. 31, 2002

(54) Novel gene encoding a sodium channel beta-3 subunit protein

(75) Inventors: Rory A.J. Curtis, Southborough, MA (US); M. Alexandra Glucksmann, Lexington, MA (US)

Correspondence Address: Jill E. Uhl, MILLENNIUM PHARMACEUTICALS, INC., 75 Sidney Street, Cambridge, MA, 02139, US

(73) Assignee: Millennium Pharmaceuticals, Inc., a Delaware corporation

(21) Appl. No.: 10/029,191

(22) Filed: Dec. 20, 2001

Related U.S. Application Data
(63) Continuation of application No. 09/569,978, filed on May 12, 2000, now abandoned.
(60) Provisional application No. 60/134,198, filed on May 14, 1999.

(51) Int. Cl. G01N 33/53; G01N 33/567; C07H 21/04; C07K 14/435; C12P 21/02; C12N 5/06

(52) U.S. Cl. 435/69.1; 435/320.1; 435/325; 530/350; 536/23.2

(57) ABSTRACT

The invention provides an isolated nucleic acid molecule encoding a novel rat sodium channel protein β subunit. This nucleic acid molecules encodes a transmembrane protein that bears substantially sequence similarity to mammalian sodium channel protein subunits. The invention also provides antisense nucleic acid molecules, expression vectors containing the nucleic acid molecules of the invention, host cells into which the expression vectors have been introduced, and non-human transgenic animals in which a nucleic acid molecule of the invention has been introduced or disrupted. The invention still further provides isolated polypeptides, fusion polypeptides, antigenic peptides and antibodies. Diagnostic, screening and therapeutic methods utilizing compositions of the invention are also provided. The nucleic acids and polypeptides of the present invention are useful as modulating agents in regulating a variety of cellular processes.

### CROSS REFERENCE TO RELATED APPLICATIONS

This application is entitled to priority to co-pending U.S. Provisional Patent application No. 60/134,198, filed May 14, 1999.

### BACKGROUND OF THE INVENTION

Mammalian sodium channels are integral transmembrane proteins which facilitate diffusion of sodium ions across the lipid bilayer membrane in which they are embedded. Mammalian sodium channels are composed of a large (e.g. 260 kilodalton in rat brain) pore-forming subunit designated α and one or more smaller subunits designated β (including, for example, β1 and β2 subunits). The permeability of sodium channel proteins with regard to sodium ions is mediated by both the α subunit and the β subunit(s) associated therewith.

Transmembrane flux of sodium ions is important for generation and maintenance of transmembrane action potentials which are necessary for transmission of signals along the membranes of excitable cells such as muscle and nerve cells (e.g. nerve, skeletal muscle, heart, and neuroendocrine cells). Voltage-sensitive (a.k.a. voltage-gated) sodium channels mediate the rapid influx of sodium ions during the rising phase of the action potential, and they also mediate re-polarization of membranes of excitable cells. Sodium channels thus serve physiologically to transiently increase permeability of the plasma membrane of excitable cells in response to changes in membrane potential, thereby facilitating propagation of an action potential along the cell surface.

The sodium channels of excitable cells are believed to exist in three interchangeable forms. In a resting state, the sodium channel protein(s) inhibits passage of sodium ions from one side of the membrane to the other. As the membrane potential becomes less negative, the sodium channel is ‘activated.’ In its activated state, the sodium channel permits passage of sodium ions therethrough at a much greater rate than in its resting state. Shortly after the sodium channel is activated, it becomes ‘inactivated,’ in which state passage of sodium ions is once again inhibited. The sodium channel remains in the inactivated state until the membrane becomes re-polarized. Thus, the sodium channel cannot be re-activated until the membrane potential returns to approximately the value it had when the channel was in the resting state.

Rat brain sodium channels are comprised of a pore-forming α subunit and additional β subunits (McClatchey et al., Human Mol. Genet. 1993, 2:745-749). cDNA sequences encoding rat sodium channel α subunits in brain, skeletal muscle, and cardiac muscle have been described, as have human skeletal muscle homologs. cDNAs encoding sodium channel β1 subunits have been isolated from rat, human, rabbit, and mouse tissues (Isom et al., 1992, Science, 256:839-842; McClatchey et al., supra; Belcher et al., 1996, Gene 170:285-286; Grosson et al., 1996, Mol. Brain Res. 42:222-226). A cDNA encoding rat brain sodium channel β2 subunit has been isolated as well (Isom et al., 1995, Cell 83:433-442).

Co-expression of α and β1 sodium channel subunits results in increases in the rates of activation and inactivation, a shift from slow- to fast-gating mode, hyperpolarizing shifts in the voltage dependence of activation and inactivation, and an increase in sodium channel expression (Isom et al., 1992, Science 256:839-842; Isom et al., 1995, J. Biol. Chem. 270:3306-3312; Bennett et al., 1993, FEBS Lett. 326:21-24; Patton et al., 1994, J. Biol. Chem. 269:17649-17655; Makita et al., 1994, J. Biol. Chem. 269:7571-7578). Rat brain β1 subunits can modulate various a subunit isoforms in brain, skeletal muscle, and heart tissues (Patton et al., 1994, J. Biol. Chem. 269:17649-17655; Isom et al., 1995, J. Biol. Chem. 270:3306-3312; Makita et al., 1994, J. Biol. Chem. 269:7571-7578; Cannon et al., 1993, Pflügers Arch. 423:155-157; Tong et al., 1993, Biochem. Biophys. Res. Commun. 195:679-685; Qu et al., 1995, J. Biol. Chem. “Modulation of cardiac Na⁺ channel expression in Xenopus oocytes by β1 subunits”). Sodium channel β2 subunits have been reported to be expressed only in neuronal sodium channels, wherein they form disulfide linkages with sodium channel α subunits and are believed to regulate sodium channel expression and assembly in neurons (Wollner et al., 1987, J. Biol. Chem. 262:14709-14715; Scheinman et al., 1989, J. Biol. Chem. 254:10660-10666).

Alteration of sodium channel subunit proteins has been associated with various human disorders in which sodium channel kinetics are altered (e.g. paramyotonia congenita and hyperkalemic periodic paralysis; McClatchey et al., supra), and it has furthermore been suggested that sodium channel protein abnormalities may contribute to or cause other diseases associated with generation or maintenance of membrane potential or transmembrane ion gradients (e.g. epilepsy, psychiatric diseases, and dementia; McClatchey et al., supra; Grosson et al., supra; U.S. Pat. No. 5,892,018).

Need remains in the art for discovery of novel subunits of mammalian sodium channels. Such discovery enables the skilled artisan to identify disease-associated mutations, to generate and identify molecules which affect sodium channel activity by interacting with such subunits, to treat mammals afflicted with diseases caused or antagonized by mutations in such subunits, to treat mammals afflicted with diseases that can be treated by modulating activity or expression of sodium channel subunits, to deliver nucleic acid vectors encoding wild type subunits to mammals which harbor altered subunits, and to identify isoforms of and subunits related by amino acid or nucleotide homology to such subunits. The present invention satisfies these needs by providing the cDNA and amino acid sequences of a novel rat sodium channel β3 subunit (herein designated “Alrxa94h5”).

### SUMMARY OF THE INVENTION

The present invention is based, at least in part, on the discovery of a cDNA molecule, designated clone Alrxa94h5, encoding a novel rat sodium channel protein β subunit designated a β3 subunit, based on amino acid homology with sodium channel protein β1 and β2 subunits (e.g. those described in Isom et al., 1992, Science, 256:839-842; McClatchey et al., supra; Belcher et al., 1996, Gene 170:285-286; Grosson et al., 1996, Mol. Brain Res. 42:222-226; and Isom et al., 1995, Cell 83:433-442). Alrxa94h5 was previously referred to as a sodium channel β2 subunit in U.S. provisional patent application No. 60/134,198, filed May 14, 1999). Subsequent to filing of that application, the inventors discovered that a different protein had been designated a “β2” sodium channel subunit (i.e. see Isom et al., 1995, Cell 83:433-442). In order to avoid confusion with the β2 protein referred to by Isom et al. (i.e. GENBANK® accession number U37026), Alrxa94h5 is referred to herein as a “β3” sodium channel subunit. Despite this change in nomenclature, the protein designated Alrxa94h5 and referred to as a β3 subunit herein is the same protein designated Alrxa94h5 and referred to as a β2 subunit in U.S. provisional patent application No. 60/134,198.

The present invention includes Alrxa94h5, fragments of this protein, derivatives of this protein, and variants (including allelic variants) of this protein. These proteins, fragments, derivatives, and variants are collectively referred to as polypeptides of the invention or proteins of the invention. Nucleic acid molecules encoding polypeptides of the invention are collectively referred to as nucleic acids of the invention.

The nucleic acids and polypeptides of the present invention are useful as modulating agents in regulating a variety of cellular processes. Accordingly, in one aspect, the present invention provides isolated nucleic acid molecules encoding a polypeptide of the invention or a biologically active portion thereof. The present invention also provides nucleic acid molecules which are suitable as primers or hybridization probes for the detection of nucleic acids encoding a polypeptide of the invention.

The invention also features nucleic acid molecules which are at least 40% (or 50%, 60%, 70%, 80%, 90%, 95%, or 98%) identical to the nucleotide sequence of any of SEQ ID NOs: 1, 21, 22, or a complement thereof.

The invention features nucleic acid molecules which include a fragment of at least 15 (25, 40, 60, 80, 100, 150, 200, 250, 300, 350, 400, 450, 550, 650, 700, 800, 900, 1000, 1200, 1400, 1600, 1800, 2000, 2200, 2400, 2600, 2800, 3000, or 3100) consecutive nucleotide residues of any of SEQ ID NOs: 1, 21, 22, or a complement thereof. In certain embodiments, the nucleic acid molecule includes either a fragment of at least 437 consecutive nucleotide residues of SEQ ID NO: 1 or at least 15 consecutive nucleotide residues of either of SEQ ID NOs: 21 and 22.

The invention also features nucleic acid molecules which include a nucleotide sequence encoding a protein having an amino acid sequence that is at least 50% (or 60%, 70%, 80%, 90%, 95%, or 98%) identical to the amino acid sequence of any of SEQ ID NOs: 2, 3, 4, 5, 6, 7, or a complement thereof.

In preferred embodiments, the nucleic acid molecules have the nucleotide sequence of any of SEQ ID NOs: 1, 21, or 22.

Also within the invention are nucleic acid molecules which encode a fragment of a polypeptide having the amino acid sequence of any of SEQ ID NOs: 2, 3, 4, 5, 6, or 7, the fragment including at least 8 (10, 15, 20, 25, 30, 40, 50, 75, 100, 125, 150, or 200) consecutive amino acids of any of SEQ ID NOs: 2, 3, 4, 5, 6, or 7.

The invention includes nucleic acid molecules which encode a naturally occurring allelic variant of a polypeptide comprising the amino acid sequence of any of SEQ ID NOs: 2, 3, 4, 5, 6, or 7, wherein the nucleic acid molecule hybridizes under stringent conditions to a nucleic acid molecule having a nucleic acid sequence encoding any of SEQ ID NOs: 1, 21, 22, or a complement thereof.

Also within the invention are: isolated polypeptides or proteins having an amino acid sequence that is at least about 50%, preferably 60%, 75%, 90%, 95%, or 98% identical to the amino acid sequence of any of SEQ ID NOs: 2, 3, 4, 5, 6, or 7.

Also within the invention are isolated polypeptides or proteins which are encoded by a nucleic acid molecule having a nucleotide sequence that is at least about 40%, preferably 50%, 75%, 85%, or 95% identical the nucleic acid sequence encoding any of SEQ ID NOs: 2, 3, 4, 5, 6, or 7, and isolated polypeptides or proteins which are encoded by a nucleic acid molecule consisting of the nucleotide sequence which hybridizes under stringent hybridization conditions to a nucleic acid molecule having the nucleotide sequence of any of SEQ ID NOs: 1, 21, or 22.

Also within the invention are polypeptides which are naturally occurring allelic variants of a polypeptide that includes the amino acid sequence of any of SEQ ID NOs: 2, 3, 4, 5, 6, or 7, wherein the polypeptide is encoded by a nucleic acid molecule which hybridizes under stringent conditions to a nucleic acid molecule having the nucleotide sequence of any of SEQ ID NOs: 1, 21, 22, or a complement thereof.

The invention also features nucleic acid molecules that hybridize under stringent conditions to a nucleic acid molecule having the nucleotide sequence of any of SEQ ID NOs: 1, 21, and 22, or a complement thereof. In other embodiments, the nucleic acid molecules are at least 437 (450, 500, 550, 600, 650, 700, 800, 900, 1000, 1200, 1400, 1600, 1800, 2000, 2250, 2500, 2750, 3000, or 3100) nucleotides in length and hybridize under stringent conditions to a nucleic acid molecule having the nucleotide sequence of any of SEQ ID NOs: 1, 21, and 22, or a complement thereof. In another embodiment, the nucleic acid molecules are at least 15 (25, 40, 60, 80, 100, 150, 200, 250, 300, 350, 400, 450, 550, 650, 700, 800, 900, 1000, 1200, 1400, 1600, 1800, 2000, 2200, 2400, or 2600) nucleotides in length and hybridize under stringent conditions to a nucleic acid molecule having the nucleotide sequence of any of SEQ ID NOs: 21 and 22. In some embodiments, the isolated nucleic acid molecules encode a cytoplasmic, transmembrane, or extracellular domain of a polypeptide of the invention. In other embodiments, the invention provides an isolated nucleic acid molecule which is antisense to the coding strand of a nucleic acid of the invention.

Another aspect of the invention provides vectors, e.g., recombinant expression vectors, comprising a nucleic acid molecule of the invention. In another embodiment, the invention provides isolated host cells (e.g., mammalian and non-mammalian cells) containing such a vector or a nucleic acid of the invention. The invention also provides methods for producing a polypeptide of the invention by culturing, in a suitable medium, a host cell of the invention containing a recombinant expression vector encoding a polypeptide of the invention such that the polypeptide of the invention is produced.

Another aspect of this invention features isolated or recombinant proteins and polypeptides of the invention. Preferred proteins and polypeptides possess at least one biological activity possessed by the corresponding naturally-occurring human polypeptide. An activity, a biological activity, and a functional activity of a polypeptide of the invention refers to an activity exerted by a protein or polypeptide of the invention on a responsive cell as determined in vivo, or in vitro, according to standard techniques. Such activities can be a direct activity, such as an association with or an enzymatic activity on a second protein or an indirect activity, such as a cellular processes (e.g., signaling activity) mediated by interaction of the protein with a second protein. Thus, such activities include, e.g., (1) the ability to form protein-protein interactions with a naturally-occurring membrane protein (e.g. another sodium channel protein subunit such as an a subunit or another P subunit) with which the naturally-occurring polypeptide interacts in vivo; (2) the ability to bind a ligand of the naturally-occurring polypeptide; (3) the ability to bind to an intracellular target of the naturally-occurring polypeptide. Other activities include: (1) the ability to mediate transmembrane sodium transport, (2) the ability to mediate maintenance of a transmembrane sodium ion gradient, (3) the ability to affect transmission of impulses through excitable (e.g. nerve, neuronal, and muscle) cells, (4) the ability to alleviate a symptom of a disorder associated with aberrant transmembrane sodium ion transport, and (5) the ability to be inactivated by compositions which normally inactivate the naturally-occurring polypeptide.

In one embodiment, a polypeptide of the invention has an amino acid sequence sufficiently identical to an identified domain of a polypeptide of the invention. As used herein, the term “sufficiently identical” refers to a first amino acid or nucleotide sequence which contains a sufficient or minimum number of identical or equivalent (e.g., with a similar side chain) amino acid residues or nucleotides to a second amino acid or nucleotide sequence such that the first and second amino acid or nucleotide sequences have a common structural domain and/or common functional activity. For example, amino acid or nucleotide sequences which contain a common structural domain having about 65% identity, preferably 75% identity, more preferably 85%, 95%, or 98% identity are defined herein as sufficiently identical.

In one embodiment, the isolated polypeptide of the invention lacks both a transmembrane and a cytoplasmic domain. In another embodiment, the polypeptide lacks both a transmembrane domain and a cytoplasmic domain and is soluble under physiological conditions.

The polypeptides of the present invention, or biologically active portions thereof, can be operably linked to a heterologous amino acid sequence to form fusion proteins. The invention further features antibody substances that specifically bind a polypeptide of the invention such as monoclonal or polyclonal antibodies, antibody fragments, single chain antibodies, and the like. In addition, the polypeptides of the invention or biologically active portions thereof can be incorporated into pharmaceutical compositions, which optionally include pharmaceutically acceptable carriers. These antibody substances can be made, for example, by providing a polypeptide of the invention to an immunocompetent vertebrate and thereafter harvesting blood or serum from the vertebrate.

In another aspect, the present invention provides methods for detecting the presence of the activity or expression of a polypeptide of the invention in a biological sample by contacting the biological sample with an agent capable of detecting an indicator of activity such that the presence of activity is detected in the biological sample.

In another aspect, the invention provides methods for modulating activity of a polypeptide of the invention comprising contacting a cell with an agent that modulates (inhibits or enhances) the activity or expression of a polypeptide of the invention such that activity or expression in the cell is modulated. In one embodiment, the agent is an antibody that specifically binds to a polypeptide of the invention.

In another embodiment, the agent modulates expression of a polypeptide of the invention by modulating transcription, splicing, or translation of an mRNA encoding a polypeptide of the invention. In yet another embodiment, the agent is a nucleic acid molecule having a nucleotide sequence that is antisense with respect to the coding strand of an mRNA encoding a polypeptide of the invention.

The present invention also provides methods to treat a subject having a disorder characterized by aberrant activity of a polypeptide of the invention or aberrant expression of a nucleic acid of the invention by administering an agent which is a modulator of the activity of a polypeptide of the invention or a modulator of the expression of a nucleic acid of the invention to the subject. In one embodiment, the modulator is a protein of the invention. In another embodiment, the modulator is a nucleic acid of the invention. In other embodiments, the modulator is a peptide, peptidomimetic, or other small molecule (e.g. small organic molecules).

The present invention also provides diagnostic assays for identifying the presence or absence of a genetic lesion or mutation characterized by at least one of: (i) aberrant modification or mutation of a gene encoding a polypeptide of the invention, (ii) mis-regulation of a gene encoding a polypeptide of the invention, and (iii) aberrant post-translational modification of a polypeptide of the invention wherein a wild-type form of the gene encodes a polypeptide having the activity of the polypeptide of the invention.

In another aspect, the invention provides a method for identifying a compound that binds to or modulates the activity of a polypeptide of the invention. In general, such methods entail measuring a biological activity of the polypeptide in the presence and absence of a test compound and identifying those compounds which alter the activity of the polypeptide.

The invention also features methods for identifying a compound which modulates the expression of a polypeptide or nucleic acid of the invention by measuring the expression of the polypeptide or nucleic acid in the presence and absence of the compound.

In yet a further aspect, the invention provides substantially purified antibodies or fragments thereof, including non-human antibodies or fragments thereof, which antibodies or fragments specifically bind with a polypeptide having an amino acid sequence which comprises a sequence selected from the group consisting of

(i) SEQ ID NOs: 2,3,4,5,6, and7;

(ii) at least 15 amino acid residues of the amino acid sequence of SEQ ID NO: 2,3,4,5, 6,or7;

(iii) an amino acid sequence which is at least 95% identical to the amino acid sequence of SEQ ID NO: 2, 3, 4, 5, 6, or 7, wherein the percent identity is determined using the ALIGN program of the GCG software package with a PAM120 weight residue table, a gap length penalty of 12, and a gap penalty of 4; and

(iv) an amino acid sequence which is encoded by a nucleic acid molecule which hybridizes with a nucleic acid having a sequence selected from the group consisting of SEQ ID NOs: 1, 21, and 22 under conditions of hybridization of 6×SSC (standard saline citrate) at 45° C. and washing in 0.2×SSC, 0.1% SDS at 65° C.

In various embodiments, the substantially purified antibodies of the invention, or fragments thereof, can be human, non-human, chimeric and/or humanized antibodies.

In another aspect, the invention provides non-human antibodies or fragments thereof, which antibodies or fragments specifically bind with a polypeptide having an amino acid sequence which comprises a sequence selected from the group consisting of:

(i) SEQ ID NOs: 2, 3, 4, 5, 6, and 7;

(ii) at least 15 amino acid residues of the amino acid sequence of SEQ ID NO: 2, 3, 4, 5, 6, or 7;

(iii) an amino acid sequence which is at least 95% identical to the amino acid sequence of SEQ ID NO: 2, 3, 4, 5, 6, or 7, wherein the percent identity is determined using the ALIGN program of the GCG software package with a PAM120 weight residue table, a gap length penalty of 12, and a gap penalty of 4; and

(iv) an amino acid sequence which is encoded by a nucleic acid molecule which hybridizes with a nucleic acid having a sequence selected from the group consisting of SEQ ID NOs: 1, 21, and 22 under conditions of hybridization of 6×SSC (standard saline citrate) at 45° C. and washing in 0.2×SSC, 0.1% SDS at 65° C.

Such non-human antibodies can be goat, mouse, sheep, horse, chicken, rabbit, or rat antibodies. Alternatively, the non-human antibodies of the invention can be chimeric and/or humanized antibodies. In addition, the non-human antibodies of the invention can be polyclonal antibodies or monoclonal antibodies.

In still a further aspect, the invention provides monoclonal antibodies or fragments thereof, which antibodies or fragments specifically bind with a polypeptide having an amino acid sequence which comprises a sequence selected from the group consisting of:

(i) SEQ ID NOs: 2, 3, 4, 5, 6, and 7;

(ii) at least 15 amino acid residues of the amino acid sequence of SEQ ID NO: 2,3,4, 5,6,or7;

(iii) an amino acid sequence which is at least 95% identical to the amino acid sequence of SEQ ID NO: 2, 3, 4, 5, 6, or 7, wherein the percent identity is determined using the ALIGN program of the GCG software package with a PAM120 weight residue table, a gap length penalty of 12, and a gap penalty of 4; and

(iv) an amino acid sequence which is encoded by a nucleic acid molecule which hybridizes with a nucleic acid having a sequence selected from the group consisting of SEQ ID NOs: 1, 21, and 22 under conditions of hybridization of 6×SSC (standard saline citrate) at 45° C. and washing in 0.2×SSC, 0. 1% SDS at 65° C.

The monoclonal antibodies can be human, humanized, chimeric and/or non-human antibodies.

In a particularly preferred embodiment, the antibody substance of the invention specifically binds with an extracellular domain of Alrxa94h5.

Any of the antibody substances of the invention can be conjugated with a therapeutic moiety or with a detectable substance. Non-limiting examples of detectable substances that can be conjugated with the antibody substances of the invention include an enzyme, a prosthetic group, a fluorescent material (i.e. a fluorophore), a luminescent material, a bioluminescent material, and a radioactive material (e.g. a radionuclide or a substituent comprising a radionuclide).

The invention also provides a kit containing an antibody substance of the invention conjugated with a detectable substance, and instructions for use. Still another aspect of the invention is a pharmaceutical composition comprising an antibody substance of the invention and a pharmaceutically acceptable carrier. In preferred embodiments, the pharmaceutical composition contains an antibody substance of the invention, a therapeutic moiety (preferably conjugated with the antibody substance), and a pharmaceutically acceptable carrier.

Other features and advantages of the invention will be apparent from the following detailed description and claims.

### BRIEF DESCRIPTION OF THE DRAWINGS

FIG. 1, comprising FIGS. 1A, 1B, and 1C, lists the nucleotide sequence (SEQ ID NO: 1; including the stop codon) of a cDNA encoding the rat sodium channel β3 protein described herein (i.e. the protein designated Alrxa94h5). The open reading frame (ORF; residues 78-725; SEQ ID NO: 21) of the cDNA is indicated by nucleotide triplets.

FIG. 2 lists the predicted amino acid sequence (SEQ ID NO: 2) of the rat sodium channel β3 protein described herein (i.e. Alrxa94h5), as deduced from the cDNA listed in FIG. 1. The singly underlined region is the predicted signal sequence, and the doubly underlined region is the predicted transmembrane domain of the protein.

FIG. 3 is an alignment of the amino acid sequence (SEQ ID NO: 2) of Alrxa94h5 (“rat”) with the amino acid sequence of a rabbit sodium channel β1 subunit precursor (SwissProt Accession No. P53788; SEQ ID NO: 20). In this alignment (pam120.mat scoring matrix, gap penalties −12/−4), the proteins are 48.0% identical. Identical amino acid residues are indicated by a “:” between the aligned amino acids. Similar amino acid residues are indicated by a “.” between the aligned amino acids.

FIG. 4 is a series of plots produced by computerized analysis of the amino acid sequence of the protein of the invention. Regions of the protein which are predicted to assume alpha helix (A), beta sheet (C), turn (E), and random coil (G) configurations by the Garnier-Robson method are indicated, as are regions predicted to assume alpha helix (B), beta sheet (D), and turn (F) configurations by the Chou-Fasman method. FIG. 4 includes a Kyte-Doolittle hydrophilicity plot (H) of the protein of the invention, wherein relatively hydrophilic regions are above the horizontal axis (value=0) and relatively hydrophobic regions are below the horizontal axis. FIG. 4 further includes indications of amphipathic regions of the protein which are predicted to assume alpha (I), beta (J), and flexible (K) configurations by the methods of Eisenberg (I and J) and Karplus-Schulz (K). FIG. 4 also includes plots of the antigenic index, as calculated by the method of Jameson-Wolf (L) and the surface probability, as calculated by the method of Emini (M).

FIG. 5, comprising FIGS. 5A, 5B, and 5C, is an alignment of the nucleotide sequence (SEQ ID NO: 1) of the cDNA clone herein designated Alrxa94h5 (“RN”) with the nucleotide sequence (SEQ ID NO: 23) of a cDNA (“OC”; GenBank Accession No. OCU35382) encoding a rabbit sodium channel β1 subunit. Vertical bars indicate identical nucleotide residues.

FIG. 6, comprising FIGS. 6A and 6B, is a pair of graphs which depict voltage-dependent activation and inactivation curves, respectively, of transformed tsA-201 cells, as described herein. FIG. 6A depicts the mean voltage dependence of activation for transformed cells. FIG. 6B depicts the mean voltage dependence of inactivation for transformed cells subjected to a 20 millisecond conditioning pre-pulse. In both figures, data collected using cells transformed with sodium channel α subunit alone are indicated with open circles; data collected using cells transformed with sodium channel α and β3 subunits are indicated with filled circles; data collected using cells transformed with sodium channel α and β2 subunits are indicated with filled squares; data collected using cells transformed with sodium channel α, β2, and β3 subunits are indicated with filled triangles.

FIG. 7, comprising FIGS. 7A and 7B, is a pair of sodium current tracings obtained from whole-cell voltage clamp electrode experiments described herein. In FIG. 7A, the upper tracing corresponds to data collected using cells transformed with sodium channel α subunit alone, and the lower tracing corresponds to data collected using cells transformed with sodium channel α and β3 subunits. In FIG. 7B, the upper tracing corresponds to data collected using cells transformed with sodium channel α and β2 subunits, and the lower tracing corresponds to data collected using cells transformed with sodium channel α, β2, and β3 subunits. In each of these two figures, the horizontal scale bar represents 5 milliseconds.

### DETAILED DESCRIPTION OF THE INVENTION

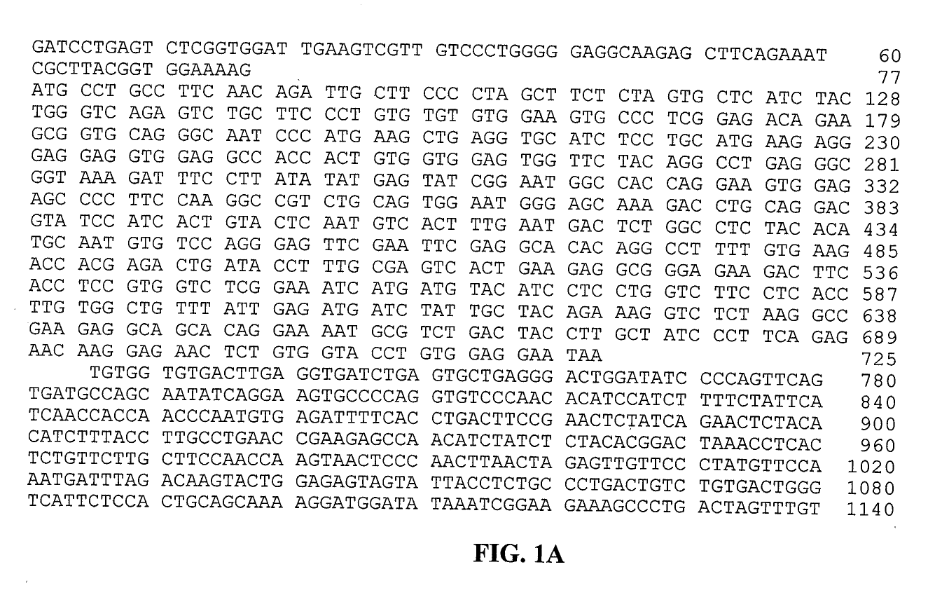

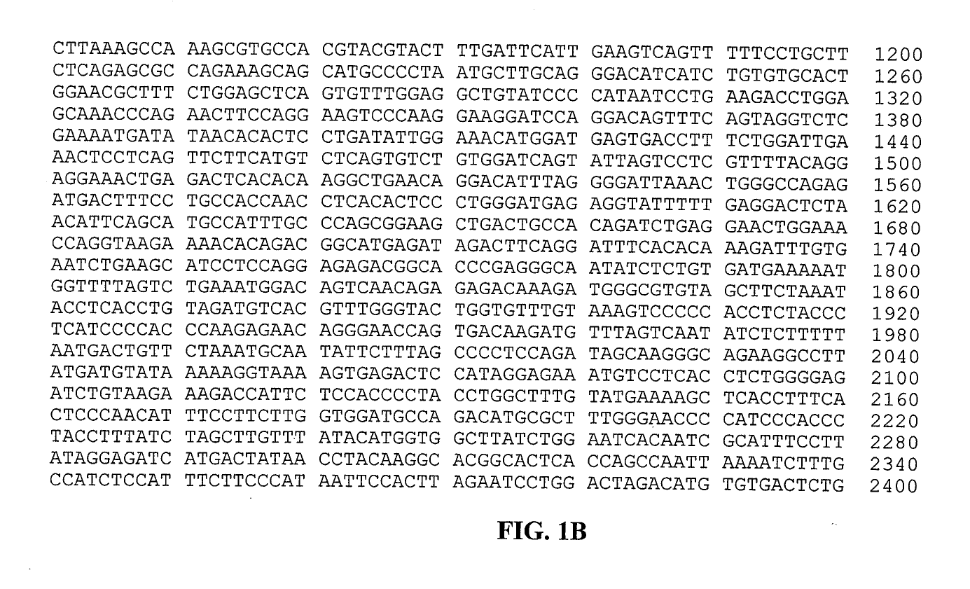

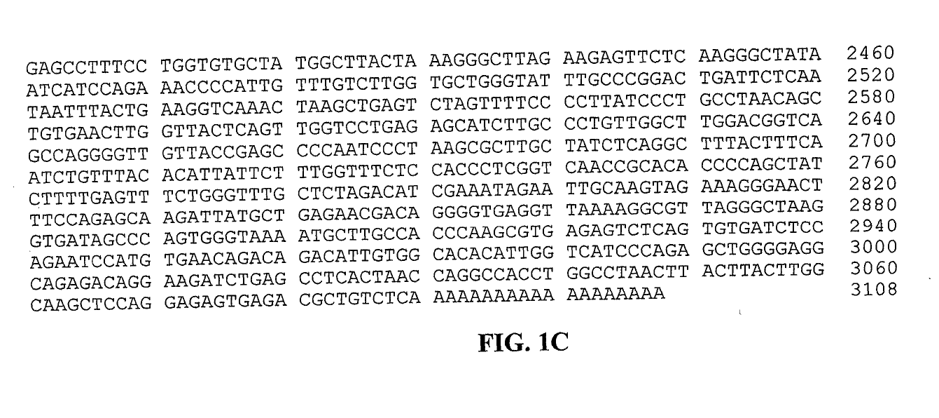

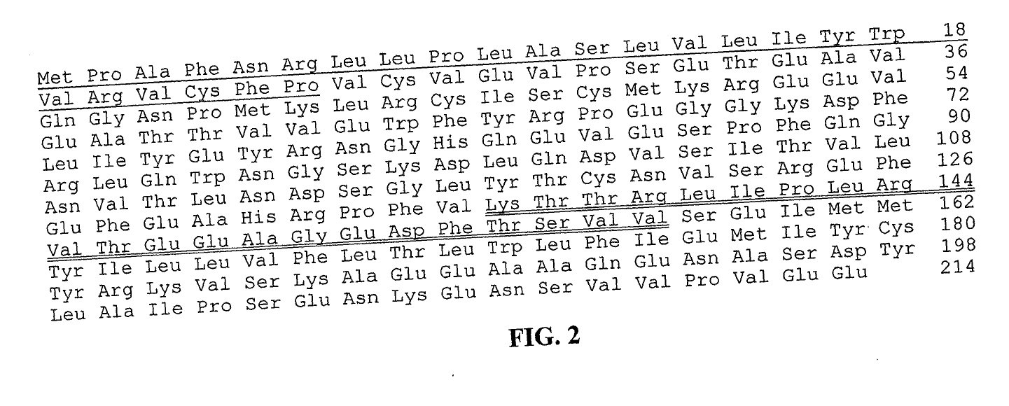

The present invention is based, at least in part, on the discovery of a cDNA molecule encoding a novel rat protein herein designated Alrxa94h5. The cDNA clone encoding Alrxa94h5 was obtained from a rat dorsal root ganglia cDNA library. The nucleotide sequence of the cDNA encoding Alrxa94h5 is listed in FIG. 1 (SEQ ID NO: 1). The amino acid sequence of Alrxa94h5 is listed in FIG. 2 (SEQ ID NO: 2).

Computerized sequence analysis of Alrxa94h5 using amino acid sequence comparison software (comparing the amino acid sequence of Alrxa94h5 with the information in the PROSITE database {rel. 12.2; Feb, 1995} and the Hidden Markov Models database {Rel. PFAM 3.3}) indicated that the protein has a cleavable signal sequence at amino acid residues 1 to about 24 (SEQ ID NO: 3), and that the mature protein (i.e. amino acid residues about 25 to 215; SEQ ID NO: 4) has an extracellular amino-terminal domain comprising about amino acid residues 25 to 135 (SEQ ID NO: 5), a transmembrane domain comprising about amino acid residues 136 to 157 (SEQ ID NO: 6), and an intracellular carboxyl-terminal domain comprising about residues 158 to 215 (SEQ ID NO: 7). Potential post-translational modification sites identified by the software are listed in Table I.

TABLE I

| | Amino Acid | Amino Acid | SEQ ID |
|---|---|---|---|
| Type of Potential Modification Site | Residues | Sequence | NO: |
| N-glycosylation site | 95 to 98 | NGSK | 8 |
| | 109 to 112 | NVTL | 9 |
| | 113 to 116 | NDSG | 10 |
| | 121 to 124 | NVSR | 11 |
| | 194 to 197 | NASD | 12 |
| cAMP- or cGMP-dependent | 182 to 185 | RKVS | 13 |
| protein kinase phosphorylation | | | |
| site | | | |
| Protein kinase C phosphorylation | 137 to 139 | TTR | 14 |
| site | | | |
| Casein kinase II phosphorylation | 31 to 34 | SETE | 15 |
| site | 58 to 61 | TVVE | 16 |
| | 111 to 114 | TLND | 17 |
| | 185 to 188 | SKAE | 18 |
| N-myristoylation site | 116 to 121 | GLYTCN | 19 |

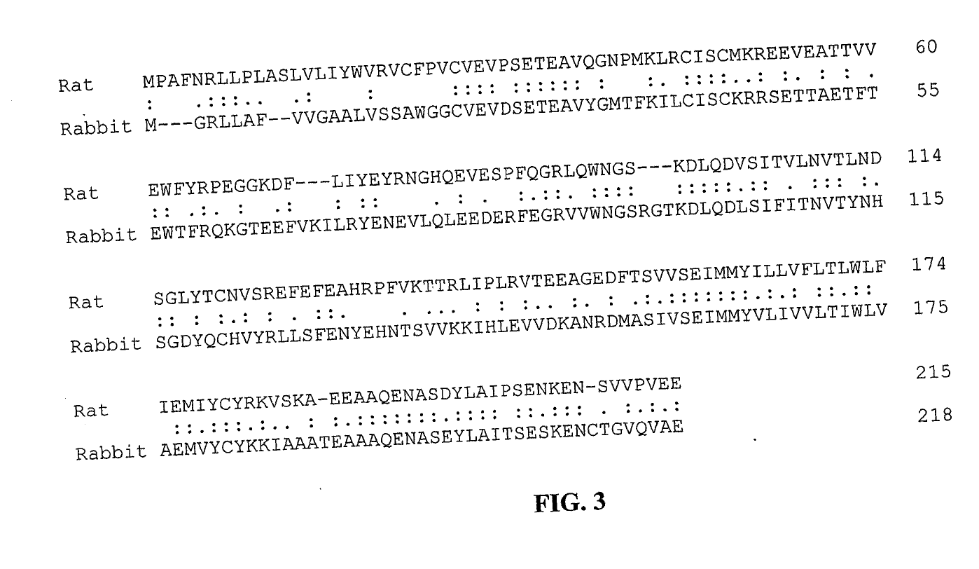

Alrxa94h5 was determined to be a sodium channel β3 subunit because it exhibited significant sequence and structural similarity with rabbit, rat, human, and mouse sodium channel β1 subunits. By way of example, an alignment of the sequences of Alrxa94h5 and the rabbit (Oryctolagus cuniculus) sodium channel β1 subunit designated P53788 in the SwissProt database is shown in FIG. 3.

In situ hybridization experiments indicated that mRNA corresponding to the cDNA encoding Alrxa94h5 is expressed in neurons of the tissues listed in Table II.

TABLE II

| animal | tissue |
|---|---|
| rat | brain |
| | dorsal horn of the spinal cord |
| | laminum 10 of the spinal cord |
| monkey | brain |
| | dorsal horn of the spinal cord |
| | laminum 10 of the spinal cord |
| | superior cervical ganglion (sympathetic |
| | neurons) |
| | a sub-population of dorsal root ganglion |
| | sensory neurons |
| human | brain |

Within brain tissues obtained from these animals, in situ hybridization analysis indicated that this gene is expressed at high levels in CA layers of hippocampus, in the subiculum, and in cerebellar Prukinje cells. In the cortex, expression is most prominent in layers I and II, with lower levels of expression observed in layers IV-VI. Low levels of expression occur in the striatum. In the spinal cord, the β3 subunit is expressed primarily in grey matter regions that are believed to be involved in nociceptive processing (i.e. laminae I, II, and V, and around the central canal). This subunit does not appear to be expressed in motor neurons. In the peripheral nervous system, expression of the β3 subunit can be detected in neuronal populations that are involved in nociception. In dorsal root ganglia, expression is restricted to neurons of small and medium size, and there is widespread expression of β3 subunit in sympathetic neurons of the superior cervical ganglion. Large proprioceptive neurons in the dorsal root ganglion did not appear to express the β3 subunit.

The invention thus includes purified Alrxa94h5 protein, both in the form of the immature 215 amino acid residue protein (SEQ ID NO: 2) and in the form of the mature 191 amino acid residue protein (SEQ ID NO: 4). The mature Alrxa94h5 protein can be synthesized without the signal sequence polypeptide at the amino terminus thereof, or it can be synthesized by generating immature Alrxa94h5 protein and cleaving the signal sequence therefrom.

In addition to full length mature and immature Alrxa94h5 protein, the invention includes fragments, derivatives, and variants of Alrxa94h5 protein. These proteins, fragments, derivatives, and variants are collectively referred to herein as polypeptides of the invention or proteins of the invention.

The invention also includes nucleic acid molecules which encode a polypeptide of the invention. Such nucleic acids include, for example, a DNA molecule having the nucleotide sequence SEQ ID NO: 1 or some portion thereof, such as the portion which encodes mature or immature Alrxa94h5 protein. These nucleic acids are collectively referred to as nucleic acids of the invention.

Alrxa94h5 proteins and nucleic acid molecules encoding them comprise a family of molecules having certain conserved structural and functional features. As used herein, the term “family” is intended to mean two or more proteins or nucleic acid molecules having a common structural domain and having sufficient amino acid or nucleotide sequence identity as defined herein. Family members can be from either the same or different species. For example, a family can comprise two or more proteins of human origin, or can comprise one or more proteins of human origin and one or more of non-human origin. Members of the same family also have common structural domains.

Also included within the scope of the invention are Alrxa94h5 proteins having a signal sequence. As used herein, a signal sequence includes a peptide of at least about 10 amino acid residues in length which occurs at the amino terminus of membrane-bound proteins and which contains at least about 45% hydrophobic amino acid residues such as alanine, leucine, isoleucine, phenylalanine, proline, tyrosine, tryptophan, or valine. In a preferred embodiment, a signal sequence contains at least about 10 to 35 amino acid residues, preferably about 10 to 20 amino acid residues, and has at least about 35-60%, more preferably 40-50%, and more preferably at least about 45% hydrophobic residues. A signal sequence serves to direct a protein containing such a sequence to a lipid bilayer. Thus, in one embodiment, an Alrxa94h5 protein contains a signal sequence corresponding to amino acids 1-24 of SEQ ID NO: 2 (SEQ ID NO: 3). The signal sequence is cleaved during processing of the mature protein.

Alrxa94h5 proteins can also include an extracellular domain. The Alrxa94h5 protein extracellular domain is located from about amino acid 25 to about amino acid 215 of SEQ ID NO: 2. Within the extracellular domain, Alrxa94h5 proteins typically comprise a variety of potential post-translational modification sites, such as those descried herein in Table I. As used herein, the term “post-translational modification site” refers to a protein domain that includes about 3-10 amino acid residues, more preferably about 3-6 amino acid residues wherein the domain has an amino acid sequence which comprises a consensus sequence which is recognized and modified by a protein-modifying enzyme. Exemplary protein-modifying enzymes include amino acid glycosylases, cAMP- and cGMP-dependent protein kinases, protein kinase C, casein kinase II, myristoylases, and prenyl transferases. In one embodiment, the protein of the invention has at least one post-translational modification site that is at least 55%, preferably at least about 65%, more preferably at least about 75%, yet more preferably at least about 85%, and most preferably at least about 95% identical to one of the post-translational modification sites described herein in Table I.

In various embodiments, the protein of the invention has the amino acid sequence of either SEQ ID NO: 2 or SEQ ID NO: 4, and comprises at least one, at least two, at least three or more, or each of the post-translational modification sites described herein in Table I.

The present invention includes Alrxa94h5 proteins having a cytoplasmic domain, particularly including proteins having a carboxyl-terminal cytoplasmic domain. The Alrxa94h5 cytoplasmic domain is located from about amino acid 158 to amino acid 215 of SEQ ID NO: 2. Thus, in one embodiment, a Alrxa94h5 protein of the invention contains a cytoplasmic domain corresponding to about amino acids 158 to 215 of SEQ ID NO: 2 (SEQ ID NO: 7).

The present invention also includes Alrxa94h5 proteins having a transmembrane domain. As used herein, a “transmembrane domain” refers to an amino acid sequence having at least about 20 to 25 amino acid residues in length and which contains at least about 65-70% hydrophobic amino acid such as alanine, leucine, phenylalanine, protein, tyrosine, tryptophan, or valine. In a preferred embodiment, a transmembrane domain contains at least about 15 to 30 amino acid residues, preferably about 20-25 amino acid residues, and has at least about 60-80%, more preferably 65-75%, and more preferably at least about 70% hydrophobic residues. Thus, in one embodiment, a Alrxa94h5 protein of the invention contains a transmembrane domain corresponding to about amino acids 136 to 157 of SEQ ID NO: 2 (SEQ ID NO: 6).

A cDNA encoding at least a portion of rat Alrxa94hS protein was isolated from a rat dorsal root ganglia cDNA library. An initial clone (designated jlrxb073e09) was selected based on its sequence similarity to sodium channel protein β1 subunits. The initial clone was used to isolate a second cDNA clone (designated Alrxa94hS) which encoded full length Alrxa94hS protein. Analysis of the cDNA designated Alrxa94hS revealed that Alrxa94h5 protein has significant sequence similarity to rat, human, rabbit, and mouse sodium channel β1 subunit proteins.

The full length of the cDNA designated Alrxa94h5 (FIG. 1; SEQ ID NO: 1) is 3108 nucleotide residues. The open reading frame of this cDNA, nucleotide residues 78 to 725 of SEQ ID NO: 1 (i.e. SEQ ID NO: 21), encodes a 215 amino acid transmembrane protein (FIG. 2; SEQ ID NO: 2).

The signal peptide prediction program SIGNALP (Nielsen et al. (1997) Protein Engineering 10:1-6) predicted that Alrxa94h5 protein includes a 24 amino acid signal peptide (amino acid residues 1-24 of SEQ ID NO: 2; SEQ ID NO: 3) preceding the mature Alrxa94h5 protein (amino acid residues 25 to 1574 of SEQ ID NO: 2; SEQ ID NO: 4). Alrxa94hS protein includes an extracellular domain (amino acid residues 25 to 135 of SEQ ID NO: 2; SEQ ID NO: 5); a transmembrane (TM) domain (amino acid residues 136 to 157 of SEQ ID NO: 2; SEQ ID NO: 6); and a cytoplasmic domain (amino acid residues 158 to 215 of SEQ ID NO: 2; SEQ ID NO: 7).

Alrxa94h5 protein has several potential post-translational modification sites, as listed herein in Table I.

The predicted molecular weight of Alrxa94h5 protein without modification and prior to cleavage of the signal sequence is 24,798.53 Daltons. The predicted molecular weight of the mature Alrxa94h5 protein without modification and after cleavage of the signal sequence about 21,997.02 Daltons.

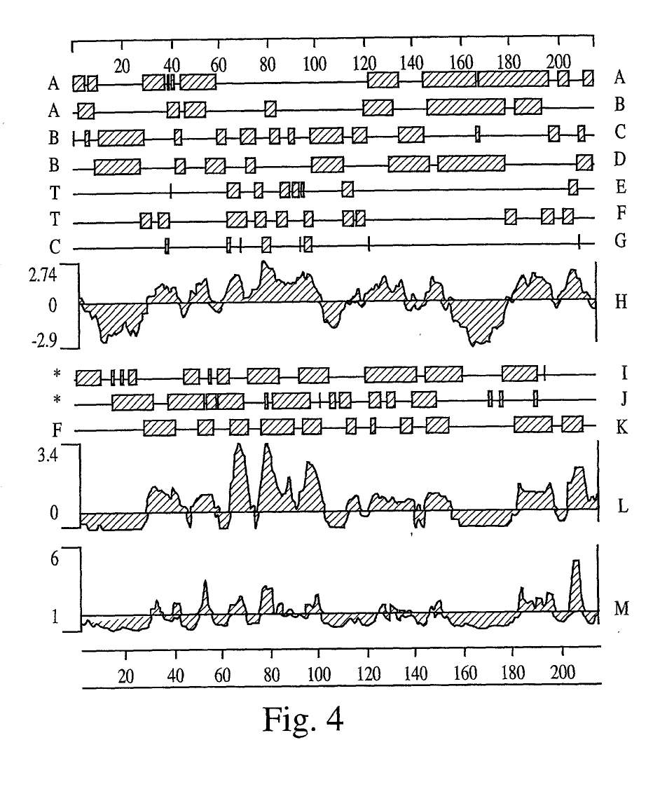

FIG. 4 depicts a hydrophilicity plot of Alrxa94h5 protein. Relatively hydrophilic regions are above the horizontal line, and relatively hydrophobic regions are below the horizontal line. The hydrophobic region which corresponds to amino acid residues 1 to 24 of SEQ ID NO: 2 is the signal sequence of Alrxa94h5 (SEQ ID NO: 3). The hydrophobic region which corresponds to amino acid residues 136-157 of SEQ ID NO: 2 is the transmembrane domain of Alrxa94h5 (SEQ ID NO: 6). As described elsewhere herein, relatively hydrophilic regions are generally located at or near the surface of a protein, and are more frequently effective immunogenic epitopes than are relatively hydrophobic regions.

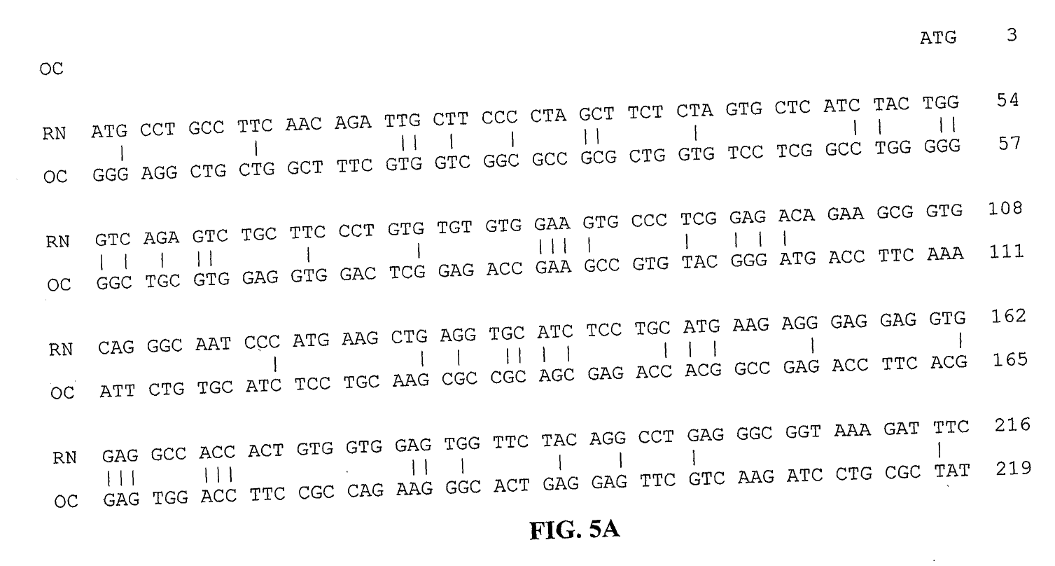

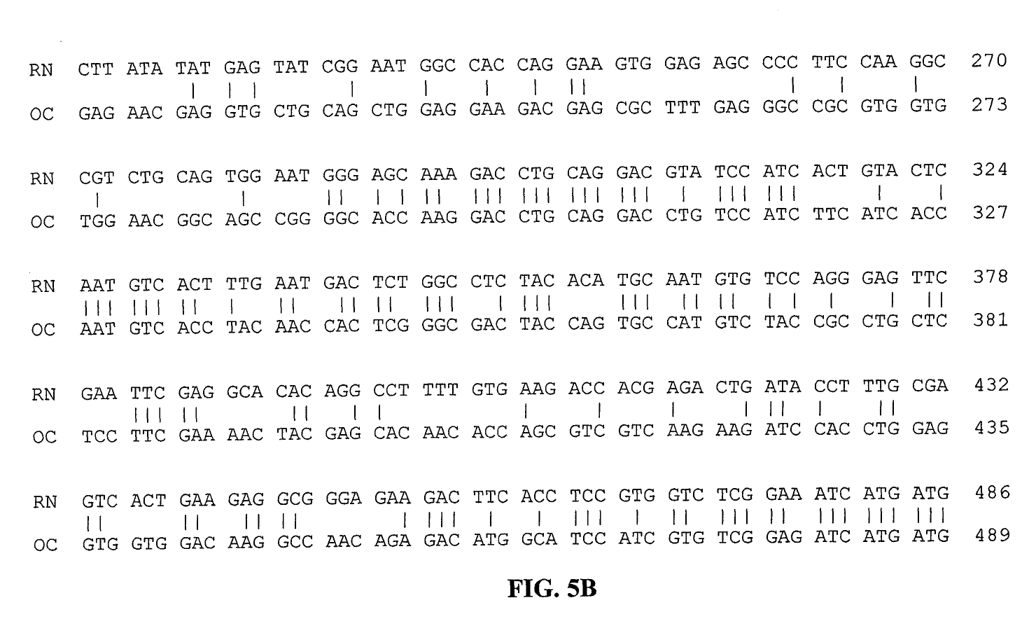

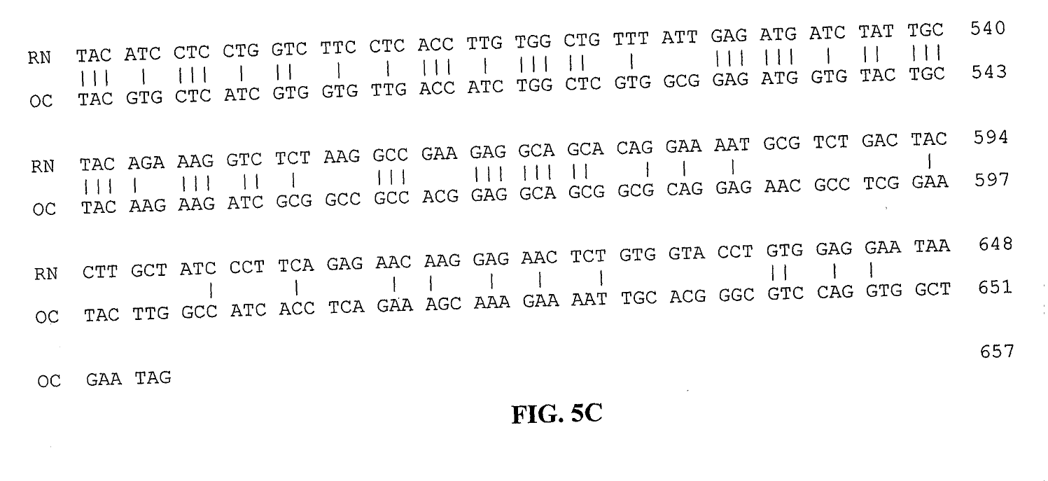

Alrxa94h5 protein exhibits considerable sequence similarity to rabbit sodium channel β1 subunit protein. FIG. 3 depicts an alignment of rabbit sodium channel β1 subunit protein (SwissProt Accession No. P53788; SEQ If) NO: 20) with Alrxa94h5 protein (SEQ ID NO: 2). In this alignment (pam120.mat scoring matrix, gap penalties −12/−4), the proteins are 48.0% identical. The cDNA encoding Alrxa94h5 protein exhibits limited similarity to various known nucleic acids. For example, the 436 nucleotide residue sequence defined by residues 2671-3047 of SEQ ID NO: 1 exhibits 100% identity with the negative (i.e. non-coding) strand of a Rattus norvegicus cDNA clone designated UI-R-E1-fv-h-12-0-UI.s1 (GenBank Accession No. AA957802). The negative strand of another R. norvegicus cDNA clone (designated UI-R-E1-fv-h-12-0-UI.s2; GenBank Accession No. AA963067) exhibits >99% identity to nucleotide residues 2633-3047 of SEQ ID NO: 1. No known nucleic acid exhibits a high degree of similarity with the portion of SEQ ID NO: 1 defined by residues 1-2632. This portion is herein designated SEQ ID NO: 22. It is believed that the known nucleic acid which is most similar to this portion is a Sugano mouse embryo cDNA clone designated 1498850 (GenBank Accession No. AI119287; SEQ ID NO: 20). The similarity of this nucleic acid (i.e. <40% identity over the length of SEQ ID NO: 20) to the sequence of the cDNA encoding Alrxa94h5 protein is illustrated in FIG. 5.

Biological Function of Alrxa94h5 Protein

Analogous to other sodium channel β subunits, Alrxa94h5 protein mediates permeability of sodium channel proteins with regard to sodium ions. Based on sequence homology, ligands of sodium channel β1 subunits are expected to function as ligands for Alrxa94h5 protein. However, Alrxa94h5 protein also has its own specific ligands and activities in addition to those reported for sodium channel β1 subunits.

Sodium channel proteins are involved in generation, alteration, and maintenance of transmembrane sodium ion gradients. Changes in transmembrane sodium ion gradients enable excitable cells (e.g. nerve cells, muscle cells, and neuronal cells of the central nervous system) to transmit impulses along their lengths. Sodium channel proteins are therefore implicated in a wide variety of normal and abnormal cellular processes which involve transmission of electrochemical impulses along biological membranes. Such processes include, for example, normal and abnormal transmission of afferent and efferent nerve impulses and normal and abnormal transmission of voluntary and involuntary muscle contractile impulses, and any disorders which result from neuronal or muscular dysfunction.

Exemplary nerve and neuronal cellular processes with which sodium channel β3 proteins such as Alrxa94h5 are involved include generation and transmission of pain and other sensory or perceptive nerve impulses, generation and maintenance of epileptic seizures, and establishment and endurance of neurodegenerative and mood disorders. Sodium channel proteins also have a role in a variety of disorders of mixed neuronal and psychological etiology including, for example, sleep disorders such as insomnia, hiccup, disorders of smell and taste, vision and eye movement disorders, hearing loss, vertigo, motor weakness, ataxias, neuropathic arthropathy disorders of the neuronal motor unit, nerve root disorders, and peripheral and hereditary neuropathies.

Exemplary muscle cell processes with which sodium channel β3 proteins such as Alrxa94h5 are involved include smooth, cardiac, striated, and skeletal muscle contraction, including normal voluntary and involuntary movements, such as heartbeat, digestion, and vascular tone. Aberrant muscular processes in which the protein of the invention have a role include, for example, arterial and renovascular hypertension, shock, cardiac insufficiency, heart failure, cardiac arrhythmias, cardiomyopathy, cardiac arrest, and skeletal muscle disorders.

Soluble forms of the sodium channel β3 protein of the invention (e.g., truncated forms of Alrxa94h5 which lack a transmembrane or cytoplasmic domain) can be used to inhibit receptor activity by interfering with the interaction between endogenous sodium channel β1, β2, or β3 subunits and their respective ligand(s).

The results presented in this application indicate that the β3 subunit of the sodium transport channel modulates sodium currents in neurons involved in nociceptive pathways. These results also indicate that agents which modulate expression or activity of this subunit can be used to treat inflammatory and neuropathic pain and disorders in which such pain is a cause or symptom.

Various aspects of the invention are described in further detail in the following subsections.

I. Isolated Nucleic Acid Molecules

One aspect of the invention pertains to isolated nucleic acid molecules that encode a polypeptide of the invention or a biologically active portion thereof, as well as nucleic acid molecules sufficient for use as hybridization probes to identify nucleic acid molecules encoding a polypeptide of the invention and fragments of such nucleic acid molecules suitable for use as PCR primers for the amplification or mutation of nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. Preferably, an “isolated” nucleic acid molecule is free of sequences (preferably protein encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of all or a portion of SEQ ID NO: 1, 21, 22, or a complement thereof, or which has a nucleotide sequence comprising one of these sequences, can be isolated using standard molecular biology techniques and the sequence information provided herein. Using a nucleic acid having a sequence comprising all or a portion of the nucleic acid sequence one or more of SEQ ID NO: 1, 21, and 22, as a hybridization probe, nucleic acid molecules of the invention can be isolated using standard hybridization and cloning techniques (e.g., as described in Sambrook et al., eds., Molecular Cloning: A Laboratory Manual, 2nd ed., Cold Spring Harbor Laboratory, Cold Spring Harbor Laboratory Press, Cold Spring Harbor, N.Y., 1989).

A nucleic acid molecule of the invention can be amplified using cDNA, mRNA or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

In another preferred embodiment, an isolated nucleic acid molecule of the invention comprises a nucleic acid molecule which is a complement of the nucleotide sequence of SEQ ID NO: 1, 21, 22, or a portion thereof. A nucleic acid molecule which is complementary to a given nucleotide sequence is one which is sufficiently complementary to the given nucleotide sequence that it can hybridize to the given nucleotide sequence thereby forming a stable duplex.

Moreover, a nucleic acid molecule of the invention can comprise only a portion of a nucleic acid sequence encoding a full length polypeptide of the invention for example, a fragment which can be used as a probe or primer or a fragment encoding a biologically active portion of a polypeptide of the invention. The nucleotide sequence determined from the cloning one gene allows for the generation of probes and primers designed for use in identifying and/or cloning homologs in other cell types, e.g., from other tissues, as well as homologs from other mammals. The probe/primer typically comprises substantially purified oligonucleotide. The oligonucleotide typically comprises a region of nucleotide sequence that hybridizes under stringent conditions to at least about 15, preferably about 25, more preferably about 50, 75, 100, 125, 150, 175, 200, 250, 300, 350, or 400 or more consecutive nucleotides of the sense or anti-sense sequence of SEQ ID NO: 1, 21, 22, or of a naturally occurring mutant of SEQ ID NO: 1,21,or22.

Probes based on the sequence of a nucleic acid molecule of the invention can be used to detect transcripts or genomic sequences encoding the same protein molecule encoded by a selected nucleic acid molecule. The probe comprises a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a nucleic acid molecule encoding the protein in a sample of cells from a subject, e.g., detecting mRNA levels or determining whether a gene encoding the protein has been mutated or deleted.

A nucleic acid fragment encoding a biologically active portion of a polypeptide of the invention can be prepared by isolating a portion of SEQ ID NO: 21 or 22, expressing the encoded portion of the polypeptide protein (e.g., by recombinant expression in vitro), and assessing the activity of the encoded portion of the polypeptide.

The invention further encompasses nucleic acid molecules that differ from the nucleotide sequence of SEQ ID NO: 1, 21, or 22 due to degeneracy of the genetic code and thus encode the same protein as that encoded by the nucleotide sequence of SEQ ID NO: 21 or 22.

In addition to the nucleotide sequences of SEQ ID NOs: 21 or 22, it will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus.

As used herein, the phrase “allelic variant” refers to a nucleotide sequence which occurs at a given locus or to a polypeptide encoded by the nucleotide sequence. For example, radiation hybrid mapping has been used to identify the location of the gene encoding Alrxa94h5 at human chromosome 11q23.3 (i.e. 1.61 centiRays from sequenced tagged site marker D11S936).

As used herein, the terms “gene” and “recombinant gene” refer to nucleic acid molecules comprising an open reading frame encoding a polypeptide of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

Moreover, nucleic acid molecules encoding proteins of the invention from other species (homologs), which have a nucleotide sequence which differs from that of the rat protein described herein are intended to be within the scope of the invention. Nucleic acid molecules corresponding to natural allelic variants and homologs of a cDNA of the invention can be isolated based on their homology with human nucleic acid molecules using the rat cDNAs, or a portion thereof, as a hybridization probe according to standard hybridization techniques under stringent hybridization conditions. For example, a cDNA encoding a soluble form of a membrane-bound protein of the invention isolated based on its hybridization to a nucleic acid molecule encoding all or part of the membrane-bound form. Likewise, a cDNA encoding a membrane-bound form can be isolated based on its hybridization to a nucleic acid molecule encoding all or part of the soluble form.

Accordingly, in another embodiment, an isolated nucleic acid molecule of the invention is at least 437 (450, 500, 550, 600, 650, 700, 800, 900, 1000, 1250, 1500, 1750, 2000, 2250, 2500, 2750, 3000, or 3108) nucleotides in length and hybridizes under stringent conditions to the nucleic acid molecule comprising the nucleotide sequence, preferably the coding sequence, of SEQ ID NO: 1, 21, or 22, or complement thereof. As used herein, the term “hybridizes under stringent conditions” is intended to describe conditions for hybridization and washing under which nucleotide sequences at least 60% (65%, 70%, preferably 75%) identical to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6′ sodium chloride/sodium citrate (SSC) at about 45° C., followed by one or more washes in 0.2′ SSC, 0.1% SDS at 50-65° C. Preferably, an isolated nucleic acid molecule of the invention that hybridizes under stringent conditions to the sequence of SEQ ID NO: 1, 21, or 22, or complement thereof, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of a nucleic acid molecule of the invention sequence that can exist in the population, the skilled artisan will further appreciate that changes can be introduced by mutation thereby leading to changes in the amino acid sequence of the encoded protein, without altering the biological activity of the protein. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species can be non-essential for activity and thus would be likely targets for alteration. Alternatively, amino acid residues that are conserved among the homologs of various species (e.g., murine and human) can be essential for activity and thus would not be likely targets for alteration.

Accordingly, another aspect of the invention pertains to nucleic acid molecules encoding a polypeptide of the invention that contain changes in amino acid residues that are not essential for activity. Such polypeptides differ in amino acid sequence from SEQ ID NO: 2 or 4, yet retain biological activity. In one embodiment, the isolated nucleic acid molecule includes a nucleotide sequence encoding a protein that includes an amino acid sequence that is at least about 40% identical, 50%, 60%, 70%, 80%, 90%, 95%, or 98% identical to the amino acid sequence of SEQ ID NO: 2 or 4.

An isolated nucleic acid molecule encoding a variant protein can be created by introducing one or more nucleotide substitutions, additions or deletions into the nucleotide sequence of SEQ ID NO: 1, 21, or 22, such that one or more amino acid substitutions, additions or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

In a preferred embodiment, a mutant polypeptide that is a variant of a polypeptide of the invention can be assayed for: (1) the ability to form protein:protein interactions with the polypeptide of the invention; (2) the ability to bind a ligand of the polypeptide of the invention (e.g. a Na⁺ ion); or (3) the ability to bind to an intracellular target protein of the polypeptide of the invention (e.g. a sodium channel protein α or β1 subunit. In yet another preferred embodiment, the mutant polypeptide can be assayed for the ability to modulate cellular proliferation, cellular migration or chemotaxis, or cellular differentiation.

The present invention encompasses antisense nucleic acid molecules, i.e., molecules which are complementary to a sense nucleic acid encoding a polypeptide of the invention, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire coding strand, or to only a portion thereof, e.g., all or part of the protein coding region (or open reading frame). An antisense nucleic acid molecule can be antisense to all or part of a non-coding region of the coding strand of a nucleotide sequence encoding a polypeptide of the invention. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′- methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a selected polypeptide of the invention to thereby inhibit expression, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an alpha-anomeric nucleic acid molecule. An alpha-anomeric nucleic acid molecule forms specific double-stranded hybrids with complementary RNA in which, contrary to the usual beta-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave mRNA transcripts to thereby inhibit translation of the protein encoded by the mRNA. A ribozyme having specificity for a nucleic acid molecule encoding a polypeptide of the invention can be designed based upon the nucleotide sequence of a cDNA disclosed herein. For example, a derivative of a Tetrahymena L-19 IVS RNA can be constructed in which the nucleotide sequence of the active site is complementary to the nucleotide sequence to be cleaved in a Cech et al. U.S. Pat. No. 4,987,071; and Cech et al. U.S. Pat. No. 5,116,742. Alternatively, an mRNA encoding a polypeptide of the invention can be used to select a catalytic RNA having a specific ribonuclease activity from a pool of RNA molecules. See, e.g., Bartel and Szostak (1993) Science 261:1411-1418.

The invention also encompasses nucleic acid molecules which form triple helical structures. For example, expression of a polypeptide of the invention can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 6(6):569-84; Helene (1992) Ann. N.Y. Acad. Sci. 660:27-36; and Maher (1992) Bioassays 14(12):807-15.

In various embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93: 14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup (1996), supra; Perry-O'Keefe et al. (1996) Proc. Natl. Acad. Sci. USA 93: 14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996), supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al. (1989) Nucleic Acids Res. 17:5973-88). PNA monomers are then coupled in a step-wise manner to produce a chimeric molecule with a 5′ PNA segment and a 3′ DNA segment (Finn et al. (1996) Nucleic Acids Res. 24(17):3357-63). Alternatively, chimeric molecules can be synthesized with a 5′ DNA segment and a 3′ PNA segment (Peterser et al. (1975) Bioorganic Med. Chem. Lett. 5:1119-11124).

In other embodiments, the oligonucleotide can include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al. (1988) Bio/Techniques 6:958-976) or intercalating agents (see, e.g., Zon (1988) Pharm. Res. 5:539-549). To this end, the oligonucleotide can be conjugated to another molecule, e.g., a peptide, hybridization triggered cross-linking agent, transport agent, hybridization-triggered cleavage agent, etc.

II. Isolated Proteins and Antibodies

One aspect of the invention pertains to isolated proteins, and biologically active portions thereof, as well as polypeptide fragments suitable for use as immunogens to raise antibodies directed against a polypeptide of the invention. In one embodiment, the native polypeptide can be isolated from cells or tissue sources by an appropriate purification scheme using standard protein purification techniques. In another embodiment, polypeptides of the invention are produced by recombinant DNA techniques. Alternative to recombinant expression, a polypeptide of the invention can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a polypeptide of the invention include polypeptides comprising amino acid sequences sufficiently identical to or derived from the amino acid sequence of the protein (e.g., the amino acid sequence shown in any of SEQ ID NOs: 2, 3, 4, 5, 6, or 7), which include fewer amino acids than the full length protein, and exhibit at least one activity of the corresponding full-length protein. Typically, biologically active portions comprise a domain or motif with at least one activity of the corresponding protein. A biologically active portion of a protein of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the invention.

Preferred polypeptides have the amino acid sequence of SEQ ID NO: 2, 3, 4, 5, 6, or 7. Other useful proteins are substantially identical (e.g., at least about 40%, preferably 50%, 60%, 70%, 80%, 90%, 95%, or 99%) to any of SEQ ID NO: 2, 3, 4, 5, 6, or 7 and retain the functional activity of the protein of the corresponding naturally-occurring protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. Id. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, only exact matches are counted.

The invention also provides chimeric or fusion proteins. As used herein, a “chimeric protein” or “fusion protein” comprises all or part (preferably biologically active) of a polypeptide of the invention operably linked to a heterologous polypeptide (i.e., a polypeptide other than the same polypeptide of the invention). Within the fusion protein, the term “operably linked” is intended to indicate that the polypeptide of the invention and the heterologous polypeptide are fused in-frame to each other. The heterologous polypeptide can be fused to the amino-terminus or the carboxyl-terminus of the polypeptide of the invention.

One useful fusion protein is a GST fusion protein in which the polypeptide of the invention is fused to the carboxyl terminus of GST sequences. Such fusion proteins can facilitate the purification of a recombinant polypeptide of the invention.

In another embodiment, the fusion protein contains a heterologous signal sequence at its amino terminus. For example, the native signal sequence of a polypeptide of the invention can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In yet another embodiment, the fusion protein is an immunoglobulin fusion protein in which all or part of a polypeptide of the invention is fused to sequences derived from a member of the immunoglobulin protein family. The immunoglobulin fusion proteins of the invention can be incorporated into pharmaceutical compositions and administered to a subject to inhibit an interaction between a ligand (soluble or membrane-bound) and a protein on the surface of a cell (receptor), to thereby suppress signal transduction in vivo. The immunoglobulin fusion protein can be used to affect the bioavailability of a cognate ligand of a polypeptide of the invention. Inhibition of ligand/receptor interaction can be useful therapeutically, both for treating proliferative and differentiative disorders and for modulating (e.g. promoting or inhibiting) cell survival. Moreover, the immunoglobulin fusion proteins of the invention can be used as immunogens to produce antibodies directed against a polypeptide of the invention in a subject, to purify ligands and in screening assays to identify molecules which inhibit the interaction of receptors with ligands.

Chimeric and fusion proteins of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the invention can be cloned into such an expression vector such that the fusion moiety is linked in-frame to the polypeptide of the invention.

A signal sequence of a polypeptide of the invention (e.g. the signal sequence in one of SEQ ID NO: 2 or 3) can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to the signal sequence itself and to the polypeptide in the absence of the signal sequence (i.e., the cleavage products). In one embodiment, a nucleic acid sequence encoding a signal sequence of the invention can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

In another embodiment, the signal sequences of the present invention can be used to identify regulatory sequences, e.g., promoters, enhancers, repressors. Since signal sequences are the most amino-terminal sequences of a peptide, it is expected that the nucleic acids which flank the signal sequence on its amino-terminal side will be regulatory sequences which affect transcription. Thus, a nucleotide sequence which encodes all or a portion of a signal sequence can be used as a probe to identify and isolate signal sequences and their flanking regions, and these flanking regions can be studied to identify regulatory elements therein.

The present invention also pertains to variants of the polypeptides of the invention. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a protein of the invention which function as either agonists (mimetics) or as antagonists can be identified by screening combinatorial libraries of mutants, e.g., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the invention from a degenerate oligonucleotide sequence. Methods for synthesizing degenerate oligonucleotides are known in the art (see, e.g., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of the coding sequence of a polypeptide of the invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, re-naturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin and Yourvan (1992) Proc. Natl. Acad. Sci. USA 89:7811-7815; Delgrave et al. (1993) Protein Engineering 6(3):327-331).

An isolated polypeptide of the invention, or a fragment thereof, can be used as an immunogen to generate antibodies using standard techniques for polyclonal and monoclonal antibody preparation. The full-length polypeptide or protein can be used or, alternatively, the invention provides antigenic peptide fragments for use as immunogens. The antigenic peptide of a protein of the invention comprises at least 8 (preferably 10, 15, 20, or 30 or more) amino acid residues of the amino acid sequence of SEQ ID NO: 2, 3, 4, 5, 6, or 7, and encompasses an epitope of the protein such that an antibody raised against the peptide forms a specific immune complex with the protein.

Preferred epitopes encompassed by the antigenic peptide are regions that are located on the surface of the protein, e.g., hydrophilic regions. FIGS. 4 is a hydrophobicity plot of the protein of the invention. This plot or similar analyses can be used to identify hydrophilic regions.

An immunogen typically is used to prepare antibodies by immunizing a suitable (i.e. immunocompetent) subject such as a rabbit, goat, mouse or other mammal or vertebrate. An appropriate immunogenic preparation can contain, for example, recombinantly expressed or chemically synthesized polypeptide. The preparation can further include an adjuvant, such as Freund's complete or incomplete adjuvant, or similar immunostimulatory agent.

Accordingly, another aspect of the invention pertains to antibodies directed against a polypeptide of the invention. The terms “antibody” and “antibody substance” as used interchangeably herein refer to immunoglobulin molecules and immunologically active portions of immunoglobulin molecules, i.e., molecules that contain an antigen binding site which specifically binds an antigen, such as a polypeptide of the invention (e.g., an epitope of a polypeptide of the invention). A molecule which specifically binds to a given polypeptide of the invention is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies can be prepared as described above by immunizing a suitable subject with a polypeptide of the invention as an immunogen. Preferred polyclonal antibody compositions are ones that have been selected for antibodies directed against (i.e. which bind specifically with) one or more polypeptides of the invention. Particularly preferred polyclonal antibody preparations are ones that contain only antibodies directed against one or more polypeptides of the invention. Particularly preferred immunogen compositions are those that contain no other human proteins such as, for example, immunogen compositions made using a non-human host cell for recombinant expression of a polypeptide of the invention. In such a manner, the only human epitope or epitopes recognized by the resulting antibody compositions raised against this immunogen will be present as part of a polypeptide or polypeptides of the invention.

The antibody titer in the immunized subject can be monitored over time by standard techniques, such as with an enzyme linked immunosorbent assay (ELISA) using immobilized polypeptide. If desired, the antibody molecules can be harvested or isolated from the subject (e.g., from the blood or serum of the subject) and further purified by well-known techniques, such as protein A chromatography to obtain the IgG fraction. Alternatively, antibodies which bind specifically with a protein or polypeptide of the invention can be selected (e.g., partially purified) or purified using chromatographic methods, such as affinity chromatography. For example, a recombinantly expressed and purified (or partially purified) protein of the invention can be produced as described herein, and covalently or non-covalently coupled with a solid support such as, for example, a chromatography column. The column thus exhibits specific affinity for antibody substances which bind specifically with the protein of the invention, and these antibody substances can be purified from a sample containing antibody substances directed against a large number of different epitopes, thereby generating a substantially purified antibody substance composition, i.e., one that is substantially free of antibody substances which do not bind specifically with the protein. By a substantially purified antibody composition is meant, in this context, that the antibody sample contains at most only 30% (by dry weight) of contaminating antibodies directed against epitopes other than those on the desired protein or polypeptide of the invention, and preferably at most 20%, yet more preferably at most 10%, and most preferably at most 5% (by dry weight) of the sample is contaminating antibodies. A purified antibody composition means that at least 99% of the antibodies in the composition are directed against the desired protein or polypeptide of the invention.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985), Monoclonal Antibodies and Cancer Therapy, Alan R. Liss, Inc., pp. 77-96) or trioma techniques. The technology for producing hybridomas is well known (see generally Current Protocols in Immunology (1994) Coligan et al. (eds.) John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions of the antibody amino acid sequence are derived from different animal species, such as those having a variable region derived from a murine monoclonal antibody and a constant region derived from a human immunoglobulin. (See, e.g., Cabilly et al., U.S. Pat. No. 4,816,567; and Boss et al., U.S. Pat. No. 4,816,397). Humanized antibodies are antibody molecules which are obtained from non-human species, which have one or more complementarity-determining regions (CDRs) derived from the non-human species, and which have a framework region derived from a human immunoglobulin molecule. (See, e.g., Queen, U.S. Pat. No.5,585,089). Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. Nos. 5,625,126; 5,633,425; 5,569,825; 5,661,016; and 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope (Jespers et al. (1994) Bio/technology 12:899-903).

An antibody directed against a polypeptide of the invention (e.g., monoclonal antibody) can be used to isolate the polypeptide by standard techniques, such as affinity chromatography or immunoprecipitation. Moreover, such an antibody can be used to detect the protein (e.g., in a cellular lysate or cell supernatant) in order to evaluate the abundance and pattern of expression of the polypeptide. The antibodies can also be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³S or ³H.

Further, an antibody substance can be conjugated with a therapeutic moiety such as a cytotoxin, a therapeutic agent, or a radioactive metal ion. Cytotoxins and cytotoxic agents include any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, and analogs or homologs of these compounds. Therapeutic agents include, antimetabolites (e.g., methotrexate, 6-mercaptopurine, 6-thioguanine, cytarabine, 5-fluorouracil, and decarbazine), alkylating agents (e.g., mechlorethamine, thioepa chlorambucil, melphalan, carmustine {BSNU}, lomustine {CCNJ}, cyclothosphamide, busulfan, dibromomannitol, streptozotocin, mitomycin C, and cis-dichlorodiamine platinum (II) (DDP) cisplatin), anthracyclines (e.g., daunorubicin {formerly daunomycin} and doxorubicin), antibiotics (e.g., dactinomycin {formerly actinomycin}, bleomycin, mithramycin, and anthramycin {AMC}), and anti-mitotic agents (e.g., vincristine and vinblastine).

The conjugates of the invention can be used to modify a biological response; the drug moiety is not to be construed as limited to classical chemical therapeutic agents. For example, the drug moiety can be a protein or polypeptide which exhibits a desired biological activity. Such proteins include, for example, toxins such as abrin, ricin A, pseudomonas exotoxin, or diphtheria toxin; proteins such as tumor necrosis factor, alpha-interferon, beta-interferon, nerve growth factor, platelet derived growth factor, tissue plasminogen activator; and biological response modifiers such as lymphokines, interleukin-1 (IL-1), interleukin-2 (IL-2), interleukin-6 (IL-6), granulocyte macrophage colony stimulating factor (GM-CSF), granulocyte colony stimulating factor (G-CSF), and other growth factors.

Techniques for conjugating a therapeutic moiety with an antibody substance are well known (see, e.g., Arnon et al., “Monoclonal Antibodies For Immunotargeting Of Drugs In Cancer Therapy”, in Monoclonal Antibodies and Cancer Therapy, Reisfeld et al., eds., pp. 243-256, Alan R. Liss, Inc., 1985; Hellstrom et al., “Antibodies For Drug Delivery”, in Controlled Drug Delivery, 2nd Ed., Robinson et al., eds., pp. 623-653, Marcel Dekker, Inc., 1987; Thorpe, “Antibody Carriers Of Cytotoxic Agents In Cancer Therapy: A Review”, in Monoclonal Antibodies '84: Biological and Clinical Applications, Pinchera et al., eds., pp. 475-506, 1985; “Analysis, Results, And Future Prospective Of The Therapeutic Use Of Radiolabeled Antibody In Cancer Therapy”, in Monoclonal Antibodies for Cancer Detection and Therapy, Baldwin et al., eds., pp. 303-316, Academic Press, 1985; and Thorpe et al., “The Preparation And Cytotoxic Properties Of Antibody-Toxin Conjugates”, Immunol. Rev. 62:119-58, 1982). Alternatively, an antibody can be conjugated with a second antibody to form an antibody heteroconjugate as described by Segal in U.S. Pat. No. 4,676,980.

Accordingly, in one aspect, the invention provides substantially purified antibodies or fragment thereof, and non-human antibodies or fragments thereof, which antibodies or fragments specifically bind with a polypeptide having an amino acid sequence which comprises a sequence selected from the group consisting of

(i) SEQ ID NOs: 2, 3, 4, 5, 6, and 7;

(ii) at least 15 amino acid residues of the amino acid sequence of SEQ ID NO: 2, 3, 4, 5, 6, or 7;

(iii) an amino acid sequence which is at least 95% identical to the amino acid sequence of SEQ ID NO: 2, 3, 4, 5, 6, or 7, wherein the percent identity is determined using the ALIGN program of the GCG software package with a PAM120 weight residue table, a gap length penalty of 12, and a gap penalty of 4; and

(iv) an amino acid sequence which is encoded by a nucleic acid molecule which hybridizes with a nucleic acid having a sequence selected from the group consisting of SEQ ID NOs: 1, 21, and 22 under conditions of hybridization of 6×SSC (standard saline citrate) at 45° C. and washing in 0.2×SSC, 0.1% SDS at 65° C.

In another aspect, the invention provides non-human antibodies or fragments thereof, which antibodies or fragments specifically bind with a polypeptide having an amino acid sequence which comprises a sequence selected from the group consisting of:

(i)SEQ ID NOs: 2, 3, 4, 5, 6, and 7;

(ii) at least 15 amino acid residues of the amino acid sequence of SEQ ID NO: 2, 3, 4, 5, 6, or 7;

(iii) an amino acid sequence which is at least 95% identical to the amino acid sequence of SEQ ID NO: 2, 3, 4, 5, 6, or 7, wherein the percent identity is determined using the ALIGN program of the GCG software package with a PAM120 weight residue table, a gap length penalty of 12, and a gap penalty of 4; and

(iv) an amino acid sequence which is encoded by a nucleic acid molecule which hybridizes with a nucleic acid having a sequence selected from the group consisting of SEQ ID NOs: 1, 21, and 22 under conditions of hybridization of 6×SSC (standard saline citrate) at 45° C. and washing in 0.2×SSC, 0.1% SDS at 65° C.

Such non-human antibodies can be goat, mouse, sheep, horse, chicken, rabbit, or rat antibodies. Alternatively, the non-human antibodies of the invention can be chimeric and/or humanized antibodies. In addition, the non-human antibodies of the invention can be polyclonal antibodies or monoclonal antibodies.

which antibodies or fragments specifically bind with a polypeptide having an amino acid sequence which comprises a sequence selected from the group consisting of:

(i) SEQ ID NOs: 2, 3, 4, 5, 6, and 7;

(ii) at least 15 amino acid residues of the amino acid sequence of SEQ ID NO: 2, 3, 4, 5, 6, or 7;

(iii) an amino acid sequence which is at least 95% identical to the amino acid sequence of SEQ ID NO: 2, 3, 4, 5, 6, or 7, wherein the percent identity is determined using the ALIGN program of the GCG software package with a PAM120 weight residue table, a gap length penalty of 12, and a gap penalty of 4; and

(iv) an amino acid sequence which is encoded by a nucleic acid molecule which hybridizes with a nucleic acid having a sequence selected from the group consisting of SEQ ID NOs: 1, 21, and 22 under conditions of hybridization of 6×SSC (standard saline citrate) at 45° C. and washing in 0.2×SSC, 0.1% SDS at 65° C.

The monoclonal antibodies can be human, humanized, chimeric and/or non-human antibodies.

The substantially purified antibodies or fragments thereof can specifically bind with a signal peptide, a secreted sequence, an extracellular domain, a transmembrane or a cytoplasmic domain cytoplasmic membrane of a polypeptide of the invention. In a particularly preferred embodiment, the substantially purified antibodies or fragments thereof, the non-human antibodies or fragments thereof, and/or the monoclonal antibodies or fragments thereof, of the invention specifically bind with a secreted sequence or with an extracellular domain of Alrxa94h5.

Any of the antibody substances of the invention can be conjugated with a therapeutic moiety or to a detectable substance. Non-limiting examples of detectable substances that can be conjugated with the antibody substances of the invention include an enzyme, a prosthetic group, a fluorescent material (i.e. a fluorophore), a luminescent material, a bioluminescent material, and a radioactive material (e.g. a radionuclide or a substituent comprising a radionuclide).

The invention also provides a kit containing an antibody substance of the invention conjugated with a detectable substance, and instructions for use. Still another aspect of the invention is a pharmaceutical composition comprising an antibody substance of the invention and a pharmaceutically acceptable carrier. In preferred embodiments, the pharmaceutical composition contains an antibody substance of the invention, a therapeutic moiety (preferably conjugated with the antibody substance), and a pharmaceutically acceptable carrier.

Still another aspect of the invention is a method of making an antibody that specifically recognizes Alrxa94h5. This method comprises immunizing a vertebrate (e.g. a mammal such as a rabbit, goat, or pig) with a polypeptide. The polypeptide used as an immunogen has an amino acid sequence that comprises a sequence selected from the group consisting of:

(i) SEQ ID NOs: 2, 3, 4, 5, 6, and 7;

(ii) at least 15 amino acid residues of the amino acid sequence of SEQ ID NO: 2, 3, 4, 5, 6, or 7;

(iii) an amino acid sequence which is at least 95% identical to the amino acid sequence of SEQ ID NO: 2, 3, 4, 5, 6, or 7, wherein the percent identity is determined using the ALIGN program of the GCG software package with a PAM120 weight residue table, a gap length penalty of 12, and a gap penalty of 4; and

(iv) an amino acid sequence which is encoded by a nucleic acid molecule which hybridizes with a nucleic acid having a sequence selected from the group consisting of SEQ ID NOs: 1, 21, and 22 under conditions of hybridization of 6×SSC (standard saline citrate) at 45° C. and washing in 0.2×SSC, 0.1% SDS at 65° C.

After immunization, a sample is collected from the vertebrate that contains an antibody that specifically recognizes the polypeptide with which the vertebrate was immunized. Preferably, the polypeptide is recombinantly produced using a non-human host cell. Optionally, an antibody substance can be further purified from the sample using techniques well known to those of skill in the art. The method can further comprise making a monoclonal antibody-producing cell from a cell of the vertebrate. Optionally, antibodies can be collected from the antibody-producing cell.

In addition, antibodies of the invention, either conjugated or not conjugated with a therapeutic moiety, can be administered together or in combination with a therapeutic moiety such as a cytotoxin, a therapeutic agent or a radioactive metal ion. The order of administration of the antibody and therapeutic moiety can vary. For example, in some embodiments, the antibody is administered concurrently (through the same or different delivery devices, e.g., syringes) with the therapeutic moiety. Alternatively, the antibody can be administered separately and prior to the therapeutic moiety. Still alternatively, the therapeutic moiety is administered separately and prior to administration of the antibody. In many embodiments, these administration regimens will be continued for days, months or years.

Another aspect of the invention is a method for inducing an immunological response in a mammal which comprises inoculating the mammal with a protein of the invention, in an amount adequate to induce an antibody and/or T cell immune response in the animal. Induction of such an immune response can be sufficient to protect the animal from one or more of the disorders described herein. Such immune responses can alternatively inhibit the onset or severity of such disorders.

Yet another aspect of the invention is a method of inducing an immunological response in a mammal. This method comprises delivering a protein of the invention (e.g., using a vector encoding the protein operably linked with sequences {e.g. promoter and/or regulatory sequences} sufficient to induce production of the protein in vivo after the vector is delivered to a subject.

The invention includes an immunological formulation (e.g., a vaccine) which, when introduced into a mammalian host, induces an immunological response in that mammal to a protein of the invention. The formulation comprises a protein of the invention, and can further comprise a suitable carrier. Because polypeptides can be degraded in the stomach, the formulation is preferably administered parenterally. The dosage of the formulation that is effective for inducing a protective immune response will depend on the specific activity of the vaccine (e.g., including the specific identity of the protein of the invention), and can be readily determined by no more than routine experimentation.

Computer Readable Means

The nucleotide or amino acid sequences of the invention are also provided in a variety of mediums to facilitate use thereof. As used herein, “provided” refers to a manufacture, other than an isolated nucleic acid or amino acid molecule, which contains a nucleotide or amino acid sequence of the present invention. Such a manufacture provides the nucleotide or amino acid sequences, or a subset thereof (e.g., a subset of open reading frames (ORFs)) in a form which allows a skilled artisan to examine the manufacture using means not directly applicable to examining the nucleotide or amino acid sequences, or a subset thereof, as they exist in nature or in purified form.

In one application of this embodiment, a nucleotide or amino acid sequence of the present invention can be recorded on computer readable media. As used herein, “computer readable media” refers to any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. This skilled artisan will readily appreciate how any of the presently known computer readable media can be used to create a manufacture comprising computer readable medium having recorded thereon a nucleotide or amino acid sequence of the present invention.

As used herein, “recorded” refers to a process for storing information on computer readable medium. The skilled artisan can readily adopt any of the presently known methods for recording information on computer readable medium to generate manufactures comprising the nucleotide or amino acid sequence information of the present invention.

A variety of data storage structures are available to a skilled artisan for creating a computer readable medium having recorded thereon a nucleotide or amino acid sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a work processing test file, formatted in commercially-available software such as WORDPERFECT™ and Microsoft WORD™, or represented in the form of an ASCII file, stored in a database application, such as DB2, SYBASE™, ORACLE™, or the like. The skilled artisan can readily adapt any number of data processor structuring formats (e.g., text file or database) in order to obtain computer readable medium having recorded thereon the nucleotide sequence information of the present invention.

By providing the nucleotide or amino acid sequences of the invention in computer readable form, the skilled artisan can routinely access the sequence information for a variety of purposes. For example, one skilled in the art can use the nucleotide or amino acid sequences of the invention in computer readable form to compare a target sequence or a target structural motif with the sequence information stored within the data storage means. Search means are used to identify fragments or regions of the sequences of the invention which match a particular target sequence or target motif.

As used herein, a “target sequence” can be any DNA or amino acid sequence of six or more nucleotides or two or more amino acids. A skilled artisan can readily recognize that the longer a target sequence is, the less likely a target sequence will be present as a random occurrence in the database. The most preferred sequence length of a target sequence is from about 10 to 100 amino acids or from about 30 to 300 nucleotide residues. However, it is recognized that commercially important fragments, such as sequence fragments involved in gene expression and protein processing, can be of shorter length.

As used herein, “a target structural motif,” or “target motif,” refers to any rationally selected sequence or combination of sequences in which the sequence(s) are chosen based on a three-dimensional configuration which is formed upon the folding of the target motif. There are a variety of target motifs know in the art. Protein target motifs include, but are not limited to, enzyme active sites and signal sequences. Nucleic acid target motifs include, but are not limited to, promoter sequences, hairpin structures and inducible expression elements (protein binding sequences).

Computer software is publicly available which allows a skilled artisan to access sequence information provided in a computer readable medium for analysis and comparison to other sequences. A variety of know algorithms are disclosed publicly and a variety of commercially available software for conducting search means are and can be used in the computer-based systems of the present invention. Examples of such software includes, but is not limited to, MacPattern (EMBL), BLASTIN and BLASTX (NCBIA).

For example, software which implements the BLAST (Altschul et al. (1990) J. of Mol. Biol. 215:403-410) and BLAZE (Brutlag et al. (1993) Comp. Chem. 17:203-207) search algorithms on a SYBASE™ system can be used to identify open reading frames (ORFs) of the sequences of the invention which contain homology to ORFs or proteins from other libraries. Such ORFs are protein-encoding fragments and are useful in producing commercially important proteins such as enzymes used in various reactions and in the production of commercially useful metabolites.

III. Recombinant Expression Vectors and Host Cells

Another aspect of the invention pertains to vectors, preferably expression vectors, containing a nucleic acid encoding a polypeptide of the invention (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein.

The recombinant expression vectors of the invention can be designed for expression of a polypeptide of the invention in prokaryotic (e.g., E. coli) or eukaryotic cells (e.g., insect cells (using baculovirus expression vectors), yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., (1988) Gene 69:301-315) and pET 1 id (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a co-expressed viral RNA polymerase (T7 gn1). This viral polymerase is supplied by host strains BL21(DE3) or HMS174(DE3) from a resident lambda prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerevisiae include pYepSec1 (Baldari et al. (1987) EMBO J. 6:229-234), pMFa (Kurjan and Herskowitz, (1982) Cell 30:933-943), pJRY88 (Schultz et al. (1987) Gene 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemnid, or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al. (Reviews—Trends in Genetics, Vol. 1(1) 1986).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells can integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

In another embodiment, the expression characteristics of an endogenous nucleic acid within a cell, cell line, or microorganism (e.g., a nucleic acid encoding Alrxa94h5, as described herein) can be modified by inserting a heterologous DNA regulatory element (i.e. one that is heterologous with respect to the endogenous gene) into the genome of the cell, stable cell line, or cloned microorganism. The inserted regulatory element can be operatively linked with the endogenous gene (e.g., that encoding Alrxa94h5) and thereby control, modulate, or activate the endogenous gene. For example, an endogenous Alrxa94h5 gene which is normally “transcriptionally silent” (i.e., a Alrxa94h5 gene which is normally not expressed, or is normally expressed only at only a very low level) can be activated by inserting a regulatory element which is capable of promoting expression of the gene in the cell, cell line, or microorganism. Alternatively, a transcriptionally silent, endogenous Alrxa94h5 gene can be activated by inserting a promiscuous regulatory element that works across cell types.

A heterologous regulatory element can be inserted into a stable cell line or cloned microorganism such that it is operatively linked with and activates expression of an endogenous Alrxa94h5 gene, using techniques, such as targeted homologous recombination, which are well known to those of skill in the art (described e.g., in Chappel, U.S. Pat. No. 5,272,071; PCT publication No. WO 91/06667, published May 16, 1991).

A host cell of the invention, such as a prokaryotic or eukaryotic host cell in culture, can be used to produce a polypeptide of the invention. Accordingly, the invention further provides methods for producing a polypeptide of the invention using the host cells of the invention. In one embodiment, the method comprises culturing the host cell of invention (into which a recombinant expression vector encoding a polypeptide of the invention has been introduced) in a suitable medium such that the polypeptide is produced. In another embodiment, the method further comprises isolating the polypeptide from the medium or the host cell.

The host cells of the invention can also be used to produce non-human transgenic animals. For example, in one embodiment, a host cell of the invention is a fertilized oocyte or an embryonic stem cell into which a sequences encoding a polypeptide of the invention have been introduced. Such host cells can then be used to create non-human transgenic animals in which exogenous sequences encoding a polypeptide of the invention have been introduced into their genome or homologous recombinant animals in which endogenous encoding a polypeptide of the invention sequences have been altered. Such animals are useful for studying the function and/or activity of the polypeptide and for identifying and/or evaluating modulators of polypeptide activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

A transgenic animal of the invention can be created by introducing nucleic acid encoding a polypeptide of the invention (or a homologue thereof) into the male pronuclei of a fertilized oocyte, e.g., by microinjection, retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to the transgene to direct expression of the polypeptide of the invention to particular cells. Methods for generating transgenic animals via embryo manipulation and microinjection, particularly animals such as mice, have become conventional in the art and are described, for example, in U.S. Pat. Nos. 4,736,866 and 4,870,009, 4,873,191, and in Hogan, Manipulating the Mouse Embryo, (Cold Spring Harbor Laboratory Press, Cold Spring Harbor, N.Y., 1986, and in Wakayama et al., 1999, Proc. Natl. Acad. Sci. USA 96:14984-14989). Similar methods are used for production of other transgenic animals. A transgenic founder animal can be identified based upon the presence of the transgene in its genome and/or expression of mRNA encoding the transgene in tissues or cells of the animals. A transgenic founder animal can then be used to breed additional animals carrying the transgene. Moreover, transgenic animals carrying the transgene can further be bred to other transgenic animals carrying other transgenes.

To create an homologous recombinant animal, a vector is prepared which contains at least a portion of a gene encoding a polypeptide of the invention into which a deletion, addition or substitution has been introduced to thereby alter, e.g., functionally disrupt, the gene. In a preferred embodiment, the vector is designed such that, upon homologous recombination, the endogenous gene is functionally disrupted (i.e., no longer encodes a functional protein; also referred to as a “knock out” vector). Alternatively, the vector can be designed such that, upon homologous recombination, the endogenous gene is mutated or otherwise altered but still encodes functional protein (e.g., the upstream regulatory region can be altered to thereby alter the expression of the endogenous protein). In the homologous recombination vector, the altered portion of the gene is flanked at its 5′ and 3′ ends by additional nucleic acid of the gene to allow for homologous recombination to occur between the exogenous gene carried by the vector and an endogenous gene in an embryonic stem cell. The additional flanking nucleic acid sequences are of sufficient length for successful homologous recombination with the endogenous gene. Typically, several kilobases of flanking DNA (both at the 5′ and 3′ ends) are included in the vector (see, e.g., Thomas and Capecchi (1987) Cell 51:503 for a description of homologous recombination vectors). The vector is introduced into an embryonic stem cell line (e.g., by electroporation) and cells in which the introduced gene has homologously recombined with the endogenous gene are selected (see, e.g., Li et al. (1992) Cell 69:915). The selected cells are then injected into a blastocyst of an animal (e.g., a mouse) to form aggregation chimeras (see, e.g., Bradley in Teratocarcinomas and Embryonic Stem Cells: A Practical Approach, Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication NOS. WO 90/11354, WO 91/01140, WO 92/0968, and WO 93/04169.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT Publication NOS. WO 97/07668 and WO 97/07669.

Nucleic acids of the invention can be delivered to a subject using a non-virus gene delivery system. Non-virus gene delivery systems include, for example, plasmid-based gene delivery systems, and are described in detail, for example, by Huang et al. (1999, Nonviral Vectors for Gene Therapy, Academic Press, San Diego, Calif.). Non-virus vectors can have advantages with respect to virus vectors, including reduced immunogenicity, lower acute toxicity, and simplicity and ease of large-scale production. Non-virus vectors can be delivered, for example, in the form of naked DNA, by bio-ballistic bombardment, and in the form of various complexes. Examples of these complexes include liposome/DNA complexes (“lipoplexes”), polymer/DNA complexes (“polyplexes”), and liposome/polymer/DNA complexes (“lipopolyplexes”). Non-virus vectors can be administered by various routes, e.g., intravenous injection, peritoneal injection, intramuscular injection, subcutaneous injection, intratracheal injection, and aerosolization.

Naked DNA (i.e., DNA not associated with, e.g., transfection-facilitating proteins, virus particles, liposomal formulations, charged lipids, or calcium phosphate), can be expressed at the site at which it is injected or otherwise applied, or it can be expressed at a remote site. For example, naked DNA can be injected directly into skeletal muscle, liver, heart muscle, or tumor tissue. For systemic administration, plasmid DNA can be protected from degradation by endonucleases during delivery from the site of administration to the site of gene expression.

Bio-ballistic bombardment, referred to colloquially as use of a ‘gene gun’, can be used to project nucleic acid-associated particles into target cells in vitro, ex vivo, or in vivo. According to this technique, DNA-coated gold particles (for example) are accelerated to a high velocity by an electric arc generated by a high voltage discharge. The method is effective for a variety of organ types, including skin, liver, muscle, spleen, and pancreas. The ‘gene gun’ transfer method does not depend on the presence of specific cell surface receptors, occurrence of a particular cell cycle status, or the size of the DNA vector. Useful ‘gene gun’ devices include the ACCELL® (PowderJect Vaccines, Inc.) and the HELIOS™ (Bio-Rad) devices. These devices create a compressed shock wave of helium gas, accelerating DNA-coated gold (or tungsten) particles to high speed, in order to impart sufficient momentum to the particles that they can penetrate a target tissue.

Lipoplexes are typically made up of three components: a cationic lipid, a neutral co-lipid, and plasmid DNA that encodes one or more genes of interest. Commonly used cationic lipids include DOTMA, DMRIE, DC-chol, DOTAP, DMRIE, DDAB, DODAB/C, DOGS, DOSPA, SAINT-n, DOSPER, DPPES, DORIE, GAP-DLRIE, and DOTIM. Dioleoyl (DO) and dimyristoyl (DM) chains are believed to be especially effective for nucleic acid delivery. Cationic lipids are typically composed of a positively-charged headgroup, a hydrophobic lipid anchor, and a linker that connects the headgroup and anchor. Catioinc lipids used in lipoplexes can be divided into two broad classes: those in which cholesterol is the lipid anchor and those which comprise diacyl chains of varying lengths and extent of saturation. The number of protonatable amines on the headgroup can affect transfection activity, multivalent headgroups being generally more active than monovalent headgroups. The linker can have a variety of chemical structures, e.g., ether, amide, carbamate, amine, urea, ester, and peptide moieties. Neutral co-lipids of lipoplexes commonly include DOPE, DOPC, and cholesterol. Generally, DOPE is used as the neutral co-lipid in combination with catioinc lipids that are based on cholesterol (e.g., DC-chol, GL-67), and cholesterol is used as the neutral co-lipid in combination with cationic lipids in which diacyl chains are the hydrophobic anchor (e.g., DOTAP, DOTIM).

Polyplexes are formed when cationic polymers are mixed with DNA. Cationic polymers used to from polyplexes are of two general types: linear polymers such as polylysine and spermine; and branched chain, spherical, or globular polycations such as polyethyleneimine and dendrimers. Lipopolyplexes are formed by incorporation of polylysine into a lipoplex to form ternary complexes. DNA can be complexed with a natural biopolymer, e.g., gelatin or chitosan, functioning as a gene carrier to form nanospheres. Such biodegradable nanospheres have several advantages, including co-encapsulation of bioactive agents (e.g., nucleic acids and drugs) and sustained release of DNA. Gelatin-DNA or chitosan-DNA nanospheres are synthesized by mixing a DNA solution with an aqueous solution of gelatin or chitosan.

The effectiveness non-virus vectors can be enhanced by conjugating the vector with one or more ligands that direct the vector either to a particular cell type or to a particular location within a cell. Antibodies and other site-specific proteins can be attached to a vector (e.g., on the surface of the vector) or incorporated in the membrane. Following injection, these vectors bind efficiently and specifically with a target site. With respect to liposomes, ligands to a cell surface receptor can be incorporated into the surface of a liposome by covalently modifying the ligand with a lipid group and adding it during the formation of liposomes.

The following classes of ligands are examples of those which can be incorporated into the non-virus DNA delivery complexes described herein in order to make them more effective for nucleic acid delivery: (1) peptides (e.g., peptides having a specific cell surface receptor such that a complex comprising the peptide is targeted to cells bearing the receptor);

(2) nucleic acids comprising a nuclear localization signal (e.g., to promote efficient entry of DNA into the nucleus);

(3) pH-sensitive ligands, to encourage endosomal escape; and

(4) steric stabilizing agents, to prevent destabilization of the complexes after introduction into the biological milieu.

Gene chemistry approaches (e.g., peptide nucleic acids) can be used to couple ligands with DNA in order to improve in vivo bioavailability and expression of the DNA. In plasmid-based, non-virus gene delivery systems, it can be useful to link a polypeptide (e.g., an antibody), nucleic acid molecule, or other compound with the gene delivery plasmid such that the polypeptide, nucleic acid molecule, or other compound remains associated with the plasmid following intracellular delivery in a manner that does not interfere with the transcriptional activity of the plasmid. This can be accomplished using an appropriate biotin-conjugated peptide nucleic acid (PNA) clamp. A sequence complementary to the biotin-conjugated PNA clamp is inserted into the gene delivery plasmid. The biotin-conjugated PNA will bind essentially irreversibly with the complementary sequence inserted into the plasmid.

A polypeptide, nucleic acid molecule, or other compound of interest can, alternatively, be conjugated with streptavidin. The streptavidin conjugate can bind with the biotin-PNA clamp bound to the plasmid. In this manner, a polypeptide, nucleic acid molecule, or other compound can be bound with a nucleic acid delivery plasmid such that the polypeptide, nucleic acid molecule or other compound remains bound to the plasmid even within a cell. The PNA clamp-binding site in the plasmid should be selected so as not to interfere with a needed promoter/enhancer or coding region, and so that it does not otherwise disrupt expression of the nucleic acid in the plasmid. In an alternative approach, a maleimide-conjugated PNA clamp is used. Polypeptides, nucleic acid molecules, and other compounds containing a free thiol residue can be conjugated directly withthe maleimide-PNA-DNA hybrid. As with the biotin-conjugation method, this conjugation does not disturb the transcriptional activity of the plasmid if the PNA-binding site is chosen to be in a region of the plasmid not essential for gene activity. Both of these approaches are described in detail by Zelphati et al. (2000, BioTechniques 28:304-315).

IV. Pharmaceutical Compositions

The nucleic acid molecules, polypeptides, and antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions for modulating the expression or activity of a polypeptide or nucleic acid of the invention. Such methods comprise formulating a pharmaceutically acceptable carrier with an agent which modulates expression or activity of a polypeptide or nucleic acid of the invention. Such compositions can further include additional active agents. Thus, the invention further includes methods for preparing a pharmaceutical composition by formulating a pharmaceutically acceptable carrier with an agent which modulates expression or activity of a polypeptide or nucleic acid of the invention and one or more additional active compounds.

The agent which modulates expression or activity can, for example, be a small molecule other than a nucleic acid, polypeptide, or antibody of the invention. For example, such small molecules include peptides, peptidomimetics, amino acids, amino acid analogs, polynucleotides, polynucleotide analogs, nucleotides, nucleotide analogs, organic or inorganic compounds (i.e. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of small molecule agents and protein or polypeptide agents depends upon a number of factors within the ken of the ordinarily skilled physician, veterinarian, or researcher. The dose(s) of these agents will vary, for example, depending upon the identity, size, and condition of the subject or sample being treated, further depending upon the route by which the composition is to be administered, if applicable, and the effect which the practitioner desires the agent to have upon the nucleic acid or polypeptide of the invention. Exemplary doses of a small molecule include milligram or microgram amounts per kilogram of subject or sample weight (e.g. about 1 microgram per kilogram to about 500 milligrams per kilogram, about 100 micrograms per kilogram to about 5 milligrams per kilogram, or about 1 microgram per kilogram to about 50 micrograms per kilogram. Exemplary doses of a protein or polypeptide include gram, milligram, or microgram amounts per kilogram of subject or sample weight (e.g., about 1 microgram per kilogram to about 5 grams per kilogram, about 100 micrograms per kilogram to about 500 milligrams per kilogram, or about 1 milligram per kilogram to about 50 milligrams per kilogram). It is furthermore understood that appropriate doses of one of these agents depend upon the potency of the agent with respect to the expression or activity to be modulated. Such appropriate doses can be determined using the assays described herein. When one or more of these agents is to be administered to an animal (e.g. a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific agent employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediamine-tetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium, and then incorporating the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches, and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes which can, e.g., be targeted to bind with virus-infected cells using a monoclonal antibody which binds specifically with a viral antigen) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

For antibodies, the preferred dosage is 0.1 mg/kg to 100 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologs, and antibodies described herein can be used in one or more of the following methods: a) screening assays; b) detection assays (e.g., chromosomal mapping, tissue typing, forensic biology); c) predictive medicine (e.g., diagnostic assays, prognostic assays, monitoring clinical trials, and pharmacogenomics); and d) methods of treatment (e.g., therapeutic and prophylactic). For example, polypeptides of the invention can to used to (i) modulate establishment or maintenance of transmembrane sodium ion gradients; (ii) modulate excitability of excitable cells; (iii) modulate the rate of signal propagation along the surface of excitable cells; and/or (iv) modulate the propensity of cells to generate a propagatable signal. The isolated nucleic acid molecules of the invention can be used to express proteins (e.g., via a recombinant expression vector in a host cell in gene therapy applications), to detect mRNA (e.g., in a biological sample) or a genetic lesion, and to modulate activity of a polypeptide of the invention. In addition, the polypeptides of the invention can be used to screen drugs or compounds which modulate activity or expression of a polypeptide of the invention as well as to treat disorders characterized by insufficient or excessive production of a protein of the invention or production of a form of a protein of the invention which has decreased or aberrant activity compared to the wild type protein. In addition, the antibodies of the invention can be used to detect and isolate a protein of the and modulate activity of a protein of the invention.

This invention further pertains to novel agents identified by the above-described screening assays and uses thereof for treatments as described herein.

A. Screening Assays

The invention provides a method (also referred to herein as a “screening assay”) for identifying modulators, i.e., candidate or test compounds or agents (e.g., peptides, peptidomimetics, small molecules or other drugs) which bind to polypeptide of the invention or have a stimulatory or inhibitory effect on, for example, expression or activity of a polypeptide of the invention.

In one embodiment, the invention provides assays for screening candidate or test compounds which bind to or modulate the activity of the membrane-bound form of a polypeptide of the invention or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds can be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or phage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310).

In one embodiment, an assay is a cell-based assay in which a cell which expresses a membrane-bound form of a polypeptide of the invention, or a biologically active portion thereof, on the cell surface is contacted with a test compound and the ability of the test compound to bind to the polypeptide determined. The cell, for example, can be a yeast cell or a cell of mammalian origin. Determining the ability of the test compound to bind to the polypeptide can be accomplished, for example, by coupling the test compound with a radioisotope or enzymatic label such that binding of the test compound to the polypeptide or biologically active portion thereof can be determined by detecting the labeled compound in a complex. For example, test compounds can be labeled with ¹²⁵I, ³⁵S, 14C, or ³H, either directly or indirectly, and the radioisotope detected by direct counting of radio-emission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In a preferred embodiment, the assay comprises contacting a cell which expresses a membrane-bound form of a polypeptide of the invention, or a biologically active portion thereof, on the cell surface with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the test compound to preferentially bind to the polypeptide or a biologically active portion thereof as compared to the known compound.

In another embodiment, the assay involves assessment of an activity characteristic of the polypeptide, wherein binding of the test compound with the polypeptide or a biologically active portion thereof alters (i.e. increases or decreases) the activity of the polypeptide.

In another embodiment, an assay is a cell-based assay comprising contacting a cell expressing a membrane-bound form of a polypeptide of the invention, or a biologically active portion thereof, on the cell surface with a test compound and determining the ability of the test compound to modulate (e.g., stimulate or inhibit) the activity of the polypeptide or biologically active portion thereof. Determining the ability of the test compound to modulate the activity of the polypeptide or a biologically active portion thereof can be accomplished, for example, by determining the ability of the polypeptide to bind to or interact with a target molecule or to transport ions (e.g. sodium ions).

Determining the ability of a polypeptide of the invention to bind to or interact with a target molecule can be accomplished by one of the methods described above for determining direct binding. As used herein, a “target molecule” is a molecule with which a selected polypeptide (e.g., a polypeptide of the invention binds or interacts with in nature, for example, a molecule on the surface of a cell which expresses the selected protein, a molecule on the surface of a second cell, a molecule in the extracellular milieu, a molecule associated with the internal surface of a cell membrane or a cytoplasmic molecule. A target molecule can be a polypeptide of the invention or some other polypeptide or protein. For example, a target molecule can be a component of a signal transduction pathway which facilitates transduction of an extracellular signal (e.g., a signal generated by binding of a compound to a polypeptide of the invention) through the cell membrane and into the cell or a second intercellular protein which has catalytic activity or a protein which facilitates the association of downstream signaling molecules with a polypeptide of the invention. Determining the ability of a polypeptide of the invention to bind to or interact with a target molecule can be accomplished by determining the activity of the target molecule. For example, the activity of the target molecule can be determined by detecting induction of a cellular second messenger of the target (e.g., an mRNA, intracellular Ca²⁺, diacylglycerol, IP3, and the like), detecting catalytic/enzymatic activity of the target on an appropriate substrate, detecting the induction of a reporter gene (e.g., a regulatory element that is responsive to a polypeptide of the invention operably linked to a nucleic acid encoding a detectable marker, e.g. luciferase), or detecting a cellular response, for example, cellular differentiation, or cell proliferation.

In yet another embodiment, an assay of the present invention is a cell-free assay comprising contacting a polypeptide of the invention or biologically active portion thereof with a test compound and determining the ability of the test compound to bind to the polypeptide or biologically active portion thereof. Binding of the test compound to the polypeptide can be determined either directly or indirectly as described above. In a preferred embodiment, the assay includes contacting the polypeptide of the invention or biologically active portion thereof with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the test compound to preferentially bind to the polypeptide or biologically active portion thereof as compared to the known compound.

In another embodiment, an assay is a cell-free assay comprising contacting a polypeptide of the invention or biologically active portion thereof with a test compound and determining the ability of the test compound to modulate (e.g., stimulate or inhibit) the activity of the polypeptide or biologically active portion thereof. Determining the ability of the test compound to modulate the activity of the polypeptide can be accomplished, for example, by determining the ability of the polypeptide to bind to a target molecule by one of the methods described above for determining direct binding. In an alternative embodiment, determining the ability of the test compound to modulate the activity of the polypeptide can be accomplished by determining the ability of the polypeptide of the invention to further modulate the target molecule. For example, the catalytic activity, the enzymatic activity, or both, of the target molecule on an appropriate substrate can be determined as previously described.

In yet another embodiment, the cell-free assay comprises contacting a polypeptide of the invention or biologically active portion thereof with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the polypeptide to preferentially bind to or modulate the activity of a target molecule.

The cell-free assays of the present invention are amenable to use of both a soluble form or the membrane-bound form of a polypeptide of the invention. In the case of cell-free assays comprising the membrane-bound form of the polypeptide, it can be desirable to utilize a solubilizing agent such that the membrane-bound form of the polypeptide is maintained in solution. Examples of such solubilizing agents include non-ionic detergents such as n-octylglucoside, n-dodecylglucoside, n-octylmaltoside, octanoyl-N-methylglucamide, decanoyl-N-methylglucamide, Triton X-100, Triton X-114, Thesit, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl-N,N-dimethyl-3-ammonio-1-propane sulfonate.

In one or more embodiments of the above assay methods of the present invention, it can be desirable to immobilize either the polypeptide of the invention or its target molecule to facilitate separation of complexed from non-complexed forms of one or both of the proteins, as well as to accommodate automation of the assay. Binding of a test compound to the polypeptide, or interaction of the polypeptide with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase fusion proteins or glutathione-S-transferase fusion proteins can be adsorbed onto glutathione SEPHAROSE™ beads (Sigma Chemical; St. Louis, Mo.) or glutathione derivatized microtiter plates, which are then combined with the test compound or the test compound and either the non-adsorbed target protein or A polypeptide of the invention, and the mixture incubated under conditions conducive to complex formation (e.g., at physiological conditions for salt and pH). Following incubation, the beads or microtiter plate wells are washed to remove any unbound components and complex formation is measured either directly or indirectly, for example, as described above. Alternatively, the complexes can be dissociated from the matrix, and the level of binding or activity of the polypeptide of the invention can be determined using standard techniques.

Other techniques for immobilizing proteins on matrices can also be used in the screening assays of the invention. For example, either the polypeptide of the invention or its target molecule can be immobilized utilizing conjugation of biotin and streptavidin. Biotinylated polypeptide of the invention or target molecules can be prepared from biotin-NHS (N-hydroxy-succinimide) using techniques well known in the art (e.g., biotinylation kit, Pierce Chemicals; Rockford, Ill.), and immobilized in the wells of streptavidin-coated 96 well plates (Pierce Chemical). Alternatively, antibodies reactive with the polypeptide of the invention or target molecules but which do not interfere with binding of the polypeptide of the invention to its target molecule can be derivatized to the wells of the plate, and unbound target or polypeptide of the invention trapped in the wells by antibody conjugation. Methods for detecting such complexes, in addition to those described above for the GST-immobilized complexes, include immunodetection of complexes using antibodies reactive with the polypeptide of the invention or target molecule, as well as enzyme-linked assays which rely on detecting an enzymatic activity associated with the polypeptide of the invention or target molecule.

In another embodiment, modulators of expression of a polypeptide of the invention are identified in a method in which a cell is contacted with a candidate compound and the expression of the selected mRNA or protein (i.e., the mRNA or protein corresponding to a polypeptide or nucleic acid of the invention) in the cell is determined. The level of expression of the selected mRNA or protein in the presence of the candidate compound is compared to the level of expression of the selected mRNA or protein in the absence of the candidate compound. The candidate compound can then be identified as a modulator of expression of the polypeptide of the invention based on this comparison. For example, when expression of the selected mRNA or protein is greater (i.e., statistically significantly greater) in the presence of the candidate compound than in its absence, the candidate compound is identified as a stimulator of the selected mRNA or protein expression. Alternatively, when expression of the selected mRNA or protein is less (i.e., statistically significantly less) in the presence of the candidate compound than in its absence, the candidate compound is identified as an inhibitor of the selected mRNA or protein expression. The level of the selected mRNA or protein expression in the cells can be determined by methods described herein.

In yet another aspect of the invention, a polypeptide of the inventions can be used as “bait proteins” in a two-hybrid assay or three hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300), to identify other proteins, which bind to or interact with the polypeptide of the invention and modulate activity of the polypeptide of the invention. Such binding proteins are also likely to be involved in the propagation of signals by the polypeptide of the inventions as, for example, upstream or downstream elements of a signaling pathway involving the polypeptide of the invention.

This invention further pertains to novel agents identified by the above-described screening assays and uses thereof for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. Accordingly, nucleic acid molecules described herein or fragments thereof, can be used to map the location of the corresponding genes on a chromosome. The mapping of the sequences to chromosomes is an important first step in correlating these sequences with genes associated with disease.

Briefly, genes can be mapped to chromosomes by preparing PCR primers (preferably 15-25 base pairs in length) from the sequence of a gene of the invention. Computer analysis of the sequence of a gene of the invention can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the gene sequences will yield an amplified fragment. For a review of this technique, see D'Eustachio et al. ((1983) Science 220:919-924).

PCR mapping of somatic cell hybrids is a rapid procedure for assigning a particular sequence to a particular chromosome. Three or more sequences can be assigned per day using a single thermal cycler. Using the nucleic acid sequences of the invention to design oligonucleotide primers, sub-localization can be achieved with panels of fragments from specific chromosomes. Other mapping strategies which can similarly be used to map a gene to its chromosome include in situ hybridization (described in Fan et al. (1990) Proc. Natl. Acad. Sci. USA 87:6223-27), pre-screening with labeled flow-sorted chromosomes, and pre-selection by hybridization to chromosome specific cDNA libraries. Fluorescence in situ hybridization (FISH) of a DNA sequence to a metaphase chromosomal spread can further be used to provide a precise chromosomal location in one step. For a review of this technique, see Verma et al. (Human Chromosomes: A Manual of Basic Techniques (Pergamon Press, New York, 1988)).

Alternatively, mapping can be performed by specifically detecting the presence of the polypeptide in members of a panel of somatic cell hybrids using cells obtained from a first species of animal from which the protein originates and cells obtained from a second species of animal and then determining which somatic cell hybrid(s) express the polypeptide and noting the chromosome(s) from the first species of animal that it contains. For examples of this technique, see Pajunen et al. (1988) Cytogenet. Cell Genet. 47:37-41 and Van Keuren et al. (1986) Hum. Genet. 74:34-40. Instead, the presence of the Alrxa94h5 polypeptide in somatic cell hybrids can be determined by assaying an activity or property of the polypeptide, for example, enzymatic activity, as described in Bordelon-Riser et al. (1979) Somatic Cell Genetics 5:597-613 and Owerbach et al. (1978) Proc. Natl. Acad. Sci. USA 75:5640-5644.

Reagents for chromosome mapping can be used individually to mark a single chromosome or a single site on that chromosome, or panels of reagents can be used for marking multiple sites and/or multiple chromosomes. Reagents corresponding to non-coding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland et al. (1987) Nature 325:783-787.

Moreover, differences in the DNA sequences between individuals affected and unaffected with a disease associated with a gene of the invention can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Furthermore, the nucleic acid sequences disclosed herein can be used to perform searches against “mapping databases”, e.g., BLAST-type search, such that the chromosome position of the gene is identified by sequence homology or identity with known sequence fragments which have been mapped to chromosomes.

2. Tissue Typing

The nucleic acid sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the nucleic acid sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The nucleic acid sequences of the invention uniquely represent portions of the human genome. Allelic variation occurs to some degree in the coding regions of these sequences, and to a greater degree in the non-coding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the non-coding regions, fewer sequences are necessary to differentiate individuals. The non-coding sequences of SEQ ID NO: 1, or 22 can comfortably provide positive individual identification with a panel of perhaps 10 to 1,000 primers which each yield a non-coding amplified sequence of 100 bases. If predicted coding sequences, such as those in SEQ ID NO: 21, are used, a more appropriate number of primers for positive individual identification would be 500-2,000.

If a panel of reagents from the nucleic acid sequences described herein is used to generate a unique identification database for an individual, those same reagents can later be used to identify tissue from that individual. Using the unique identification database, positive identification of the individual, living or dead, can be made from extremely small tissue samples.

3. Use of Partial Gene Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to non-coding regions are particularly appropriate for this use as greater numbers of polymorphisms occur in the non-coding regions, making it easier to differentiate individuals using this technique. Examples of polynucleotide reagents include the nucleic acid sequences of the invention or portions thereof, e.g., fragments derived from non-coding regions having a length of at least 20 or 30 bases.

The nucleic acid sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such probes can be used to identify tissue by species and/or by organ type.

C. Predictive Medicine

The present invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the present invention relates to diagnostic assays for determining expression of a polypeptide or nucleic acid of the invention and/or activity of a polypeptide of the invention, in the context of a biological sample (e.g., blood, serum, cells, tissue) to thereby determine whether an individual is afflicted with a disease or disorder, or is at risk of developing a disorder, associated with aberrant expression or activity of a polypeptide of the invention. The invention also provides for prognostic (or predictive) assays for determining whether an individual is at risk of developing a disorder associated with aberrant expression or activity of a polypeptide of the invention. For example, mutations in a gene of the invention can be assayed in a biological sample. Such assays can be used for prognostic or predictive purpose to thereby prophylactically treat an individual prior to the onset of a disorder characterized by or associated with aberrant expression or activity of a polypeptide of the invention.

As an alternative to making determinations based on the absolute expression level of a selected gene, determinations can be based on normalized expression levels of the gene. A gene expression level is normalized by correcting the absolute expression level of the gene (e.g. a Alrxa94h5 gene as described herein) by comparing its expression to expression of a gene for which expression is not believed to be co-regulated with the gene of interest, e.g., a housekeeping gene that is constitutively expressed. Suitable genes for normalization include housekeeping genes such as the actin gene. Such normalization allows comparison of the expression level in one sample, e.g., a patient sample, with the expression level in another sample, e.g., a sample obtained from a patient known not to be afflicted with a disease or condition, or between samples obtained from different sources.

Alternatively, the expression level can be assessed as a relative expression level. To assess a relative expression level for a gene (e.g. a Alrxa94h5 gene, as described herein), the level of expression of the gene is determined for 10 or more samples (preferably 50 or more samples) of different isolates of cells in which the gene is believed to be expressed, prior to assessing the level of expression of the gene in the sample of interest. The mean expression level of the gene detected in the large number of samples is determined, and this value is used as a baseline expression level for the gene. The expression level of the gene assessed in the test sample (i.e. its absolute level of expression) is divided by the mean expression value to yield a relative expression level. Such a method can identify tissues or individuals which are afflicted with a disorder associated with aberrant expression of a gene of the invention.

Preferably, the samples used in the baseline determination are generated either using cells obtained from a tissue or individual known to be afflicted with a disorder (e.g. a disorder associated with aberrant expression of one of the Alrxa94h5 genes) or using cells obtained from a tissue or individual known not to be afflicted with the disorder. Alternatively, levels of expression of these genes in tissues or individuals known to be or not to be afflicted with the disorder can be used to assess whether the aberrant expression of the gene is associated with the disorder (e.g., with onset of the disorder, or as a symptom of the disorder over time).

Another aspect of the invention provides methods for expression of a nucleic acid or polypeptide of the invention or activity of a polypeptide of the invention in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent).

Yet another aspect of the invention pertains to monitoring the influence of agents (e.g., drugs or other compounds) on the expression or activity of a polypeptide of the invention in clinical trials. These and other agents are described in further detail in the following sections.

1. Diagnostic Assays

An exemplary method for detecting the presence or absence of a polypeptide or nucleic acid of the invention in a biological sample involves obtaining a biological sample from a test subject and contacting the biological sample with a compound or an agent capable of detecting a polypeptide or nucleic acid (e.g., mRNA, genomic DNA) of the invention such that the presence of a polypeptide or nucleic acid of the invention is detected in the biological sample. A preferred agent for detecting mRNA or genomic DNA encoding a polypeptide of the invention is a labeled nucleic acid probe capable of hybridizing to mRNA or genomic DNA encoding a polypeptide of the invention. The nucleic acid probe can be, for example, a full-length cDNA, such as the nucleic acid of SEQ ID NO: 1, or 22, or a portion thereof, such as an oligonucleotide of at least 15, 30, 50, 100, 250 or 500 nucleotides in length and sufficient to specifically hybridize under stringent conditions to a mRNA or genomic DNA encoding a polypeptide of the invention. Other suitable probes for use in the diagnostic assays of the invention are described herein.

A preferred agent for detecting a polypeptide of the invention is an antibody capable of binding to a polypeptide of the invention, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect mRNA, protein, or genomic DNA in a biological sample in vitro as well as in vivo. For example, in vitro techniques for detection of mRNA include Northern hybridizations and in situ hybridizations. In vitro techniques for detection of a polypeptide of the invention include enzyme linked immunosorbent assays (ELISAs), Western blots, immunoprecipitations and immunofluorescence. In vitro techniques for detection of genomic DNA include Southern hybridizations. Furthermore, in vivo techniques for detection of a polypeptide of the invention include introducing into a subject a labeled antibody directed against the polypeptide. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting a polypeptide of the invention or mRNA or genomic DNA encoding a polypeptide of the invention, such that the presence of the polypeptide or mRNA or genomic DNA encoding the polypeptide is detected in the biological sample, and comparing the presence of the polypeptide or mRNA or genomic DNA encoding the polypeptide in the control sample with the presence of the polypeptide or mRNA or genomic DNA encoding the polypeptide in the test sample.

The invention also encompasses kits for detecting the presence of a polypeptide or nucleic acid of the invention in a biological sample (a test sample). Such kits can be used to determine if a subject is suffering from or is at increased risk of developing a disorder associated with aberrant expression of a polypeptide of the invention (e.g., a proliferative disorder, e.g., psoriasis or cancer). For example, the kit can comprise a labeled compound or agent capable of detecting the polypeptide or mRNA encoding the polypeptide in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for observing that the tested subject is suffering from or is at risk of developing a disorder associated with aberrant expression of the polypeptide if the amount of the polypeptide or mRNA encoding the polypeptide is above or below a normal level.

For antibody-based kits, the kit can comprise, for example: (1) a first antibody (e.g., attached to a solid support) which binds to a polypeptide of the invention; and, optionally, (2) a second, different antibody which binds to either the polypeptide or the first antibody and is conjugated to a detectable agent.

For oligonucleotide-based kits, the kit can comprise, for example: (1) an oligonucleotide, e.g., a detectably labeled oligonucleotide, which hybridizes to a nucleic acid sequence encoding a polypeptide of the invention or (2) a pair of primers useful for amplifying a nucleic acid molecule encoding a polypeptide of the invention. The kit can also comprise, e.g., a buffering agent, a preservative, or a protein stabilizing agent. The kit can also comprise components necessary for detecting the detectable agent (e.g., an enzyme or a substrate). The kit can also contain a control sample or a series of control samples which can be assayed and compared to the test sample contained. Each component of the kit is usually enclosed within an individual container and all of the various containers are within a single package along with instructions for observing whether the tested subject is suffering from or is at risk of developing a disorder associated with aberrant expression of the polypeptide.

2. Prognostic Assays

The methods described herein can furthermore be utilized as diagnostic or prognostic assays to identify subjects having or at risk of developing a disease or disorder associated with aberrant expression or activity of a polypeptide of the invention. For example, the assays described herein, such as the preceding diagnostic assays or the following assays, can be utilized to identify a subject having or at risk of developing a disorder associated with aberrant expression or activity of a polypeptide of the invention, e.g., a proliferative disorder, e.g., psoriasis or cancer, or an angiogenic disorder. Alternatively, the prognostic assays can be utilized to identify a subject having or at risk for developing such a disease or disorder. Thus, the present invention provides a method in which a test sample is obtained from a subject and a polypeptide or nucleic acid (e.g., mRNA, genomic DNA) of the invention is detected, wherein the presence of the polypeptide or nucleic acid is diagnostic for a subject having or at risk of developing a disease or disorder associated with aberrant expression or activity of the polypeptide. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant expression or activity of a polypeptide of the invention. For example, such methods can be used to determine whether a subject can be effectively treated with a specific agent or class of agents (e.g., agents of a type which decrease activity of the polypeptide). Thus, the present invention provides methods for determining whether a subject can be effectively treated with an agent for a disorder associated with aberrant expression or activity of a polypeptide of the invention in which a test sample is obtained and the polypeptide or nucleic acid encoding the polypeptide is detected (e.g., wherein the presence of the polypeptide or nucleic acid is diagnostic for a subject that can be administered the agent to treat a disorder associated with aberrant expression or activity of the polypeptide).

The methods of the invention can also be used to detect genetic lesions or mutations in a gene of the invention, thereby determining if a subject with the lesioned gene is at risk for a disorder characterized aberrant expression or activity of a polypeptide of the invention. In preferred embodiments, the methods include detecting, in a sample of cells from the subject, the presence or absence of a genetic lesion or mutation characterized by at least one of an alteration affecting the integrity of a gene encoding the polypeptide of the invention, or the mis-expression of the gene encoding the polypeptide of the invention. For example, such genetic lesions or mutations can be detected by ascertaining the existence of at least one of: 1) a deletion of one or more nucleotides from the gene; 2) an addition of one or more nucleotides to the gene; 3) a substitution of one or more nucleotides of the gene; 4) a chromosomal rearrangement of the gene; 5) an alteration in the level of a messenger RNA transcript of the gene; 6) an aberrant modification of the gene, such as of the methylation pattern of the genomic DNA; 7) the presence of a non-wild type splicing pattern of a messenger RNA transcript of the gene; 8) a non-wild type level of the protein encoded by the gene; 9) an allelic loss of the gene; and 10) an inappropriate post-translational modification of the protein encoded by the gene. As described herein, there are a large number of assay techniques known in the art which can be used for detecting lesions in a gene.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a gene (see, e.g., Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to the selected gene under conditions such that hybridization and amplification of the gene (if present) occurs, and detecting the presence or absence of an amplification product, or detecting the size of the amplification product and comparing the length to a control sample. PCR and/or LCR can be desirable to use as a preliminary amplification step in conjunction with any of the techniques used for detecting mutations described herein.

Alternative amplification methods include: self sustained sequence replication (Guatelli et al. (1990) Proc. Natl. Acad. Sci. USA 87:1874-1878), transcriptional amplification system (Kwoh, et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a selected gene from a sample cell can be identified by alterations in restriction enzyme cleavage patterns. For example, sample and control DNA is isolated, (optionally) amplified, digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,498,531) can be used to score for the presence of specific mutations by development or loss of a ribozyme cleavage site.

In other embodiments, genetic mutations can be identified by hybridizing a sample and control nucleic acids, e.g., DNA or RNA, to high density arrays containing hundreds or thousands of oligonucleotides probes (Cronin et al. (1996) Human Mutation 7:244-255; Kozal et al. (1996) Nature Medicine 2:753-759). For example, genetic mutations can be identified in two-dimensional arrays containing light-generated DNA probes as described in Cronin et al., supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the selected gene and detect mutations by comparing the sequence of the sample nucleic acids with the corresponding wild-type (control) sequence. Examples of sequencing reactions include those based on techniques developed by Maxim and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Bio/Techniques 19:448), including sequencing by mass spectrometry (see, e.g., PCT Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in a selected gene include methods in which protection from cleavage agents is used to detect mismatched bases in RNA/RNA or RNA/DNA heteroduplexes (Myers et al. (1985) Science 230:1242). In general, the technique of mismatch cleavage entails providing heteroduplexes formed by hybridizing (labeled) RNA or DNA containing the wild-type sequence with potentially mutant RNA or DNA obtained from a tissue sample. The double-stranded duplexes are treated with an agent which cleaves single-stranded regions of the duplex such as which will exist due to base pair mismatches between the control and sample strands. RNA/DNA duplexes can be treated with RNase to digest mismatched regions, and DNA/DNA hybrids can be treated with S1 nuclease to digest mismatched regions.

In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton et al. (1988) Proc. Natl. Acad. Sci. USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called DNA mismatch repair enzymes) in defined systems for detecting and mapping point mutations in cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a selected sequence, e.g., a wild-type sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in genes. For example, single strand conformation polymorphism (SSCP) can be used to detect differences in electrophoretic mobility between mutant and wild type nucleic acids (Orita et al. (1989) Proc. Natl. Acad. Sci. USA 86:2766; see also Cotton (1993) Mutat. Res. 285:125-144; Hayashi (1992) Genet. Anal. Tech. Appl. 9:73-79). Single-stranded DNA fragments of sample and control nucleic acids will be denatured and allowed to renature. The secondary structure of single-stranded nucleic acids varies according to sequence, and the resulting alteration in electrophoretic mobility enables the detection of even a single base change. The DNA fragments can be labeled or detected with labeled probes. The sensitivity of the assay can be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a ‘GC clamp’ of approximately 40 base pairs of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers can be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification can be used in conjunction with the instant invention. Oligonucleotides used as primers for specific amplification can carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent or reduce polymerase extension (Prossner (1993) Tibtech 11:238). In addition, it can be desirable to introduce a novel restriction site in the region of the mutation to create cleavage-based detection (Gasparini et al. (1992) Mol. Cell Probes 6:1). Amplification can also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

The methods described herein can be performed, for example, using pre-packaged diagnostic kits comprising at least one probe nucleic acid or antibody reagent described herein, which can be conveniently used, e.g., in clinical settings to diagnose patients exhibiting symptoms or family history of a disease or illness involving a gene encoding a polypeptide of the invention. Furthermore, any cell type or tissue, preferably peripheral blood leukocytes, in which the polypeptide of the invention is expressed can be utilized in the prognostic assays described herein.

An exemplary method for assessing the presence or absence of a protein of the invention in a biological sample involves obtaining a biological sample from a test subject and contacting the biological sample with a compound or an agent capable of detecting the protein of the invention or a nucleic acid (e.g., mRNA, genomic DNA) that encodes it. An example of an agent for detecting mRNA or genomic DNA encoding a protein of the invention is a labeled nucleic acid probe capable of hybridizing with mRNA or genomic DNA encoding Alrxa94h5. The nucleic acid probe can be, for example, a full-length Alrxa94h5 nucleic acid, such as a nucleic acid having a nucleotide sequence selected from the group consisting of SEQ ID NOs: 1, 21, and 22, or a portion thereof, such as an oligonucleotide of at least 15 (25, 40, 60, 80, 100, 150, 200, 250, 300, 350,400, 450, 550, 650, 700, 800, 900, 1000, 1200, 1400, 1600, 1800, 2000, 2200, 2400, 2600, 2800, 3000, or 3100) nucleotides in length and sufficiently homologous with one or more of SEQ ID NOs: 1, 21, and 22 that the oligonucleotide hybridizes under stringent conditions with mRNA or genomic DNA obtained from the subject if the mRNA or genomic DNA encodes Alrxa94h5. Other suitable probes for use in the diagnostic assays of the invention are described elsewhere herein.

An agent for detecting a protein of the invention can be an antibody capable of binding with the protein, preferably an antibody conjugated with a detectable label.

The term “biological sample” includes tissues, cells, biological fluids, and stool samples obtained from a subject, as well as tissues, cells and fluids present within a subject. That is, the detection methods of the invention can be used to detect Alrxa94h5 mRNA, protein, or genomic DNA in a biological sample in vitro or in vivo. For example, in vitro techniques for detecting a protein of the invention, or an mRNA encoding it include Northern hybridization and in situ hybridization. In vitro techniques for detecting a protein of the invention include enzyme linked immunosorbent assay (ELISA) techniques, Western blot, immunoprecipitation, and immunofluorescence. In vitro techniques for detecting genomic DNA encoding Alrxa94h5 include, for example, Southern hybridization. Furthermore, in vivo techniques for detecting a protein of the invention include introducing into a subject a labeled antibody substance which binds specifically with Alrxa94h5. For example, the antibody substance can be labeled by conjugating it with a radioactive marker, the presence and location of which can be detected in a subject using standard imaging techniques.

Stool samples, for example, can be analyzed using various in vitro techniques, including techniques involving analysis of DNA, RNA, or protein that is present in the sample (see, e.g., Machiels et al., 2000, BioTechniques 28:286-290). In one embodiment, the biological sample comprises protein molecules which originated in the test subject. Alternatively, the biological sample can contain mRNA molecules or genomic DNA molecules which originated in the test subject. Another example of a biological sample is a peripheral blood leukocyte sample isolated by conventional means from a subject. In another embodiment, the methods further involve obtaining a control biological sample from a control subject, contacting the control sample with a compound or agent capable of detecting a protein or nucleic acid of the invention, and comparing the presence of the protein or nucleic acid of the invention in the control sample with the presence of protein or nucleic acid of the invention in the test sample.

The invention includes kits for detecting the presence of protein or nucleic acid of the invention in a biological sample, such as one obtained from a human patient. Such kits can be used to determine if a patient is afflicted with or is at risk for developing a disorder associated with aberrant expression of the gene encoding Alrxa94h5. For example, the kit can comprise a labeled compound or agent capable of detecting a protein or nucleic acid of the invention in a biological sample. The kit can further comprise a means for determining the amount of protein or nucleic acid of the invention in the sample. Kits can also include an instructional material which describes that the tested subject may be suffering from or is at risk for developing a disorder associated with aberrant expression of the gene encoding Alrxa94h5 if the amount of the protein or nucleic acid of the invention in a sample obtained from the subject is above or below a specified value or above or below a normal level.

The methods described herein can be used to detect genetic lesions or mutations in a gene encoding Alrxa94h5. Detection of such lesions or mutations can be correlated with occurrence in a subject comprising the lesioned gene of a disorder characterized by aberrant expression of Alrxa94h5. Detection of these lesions or mutations in a subject can also be correlated with an enhanced risk for developing such a disorder. Lesions in a gene encoding Alrxa94h5 can include gene alterations which affecting the integrity of a gene encoding Alrxa94h5 and alterations which affect expression of the gene encoding Alrxa94h5. For example, such lesions can be detected by ascertaining the existence of at least one of

1) deletion of one or more nucleotides from a gene encoding Alrxa94h5;

2) addition of one or more nucleotides to a gene encoding Alrxa94h5;

3) substitution of one or more nucleotides of a gene encoding Alrxa94h5;

4) chromosomal rearrangement involving a gene encoding Alrxa94h5;

5) alteration of the level of a messenger RNA transcript of a gene encoding Alrxa94h5;

6) aberrant modification of a gene encoding Alrxa94h5 (e.g., modification of the methylation pattern of genomic DNA);

7) presence of a non-wild type splicing pattern of a messenger RNA transcript of a gene encoding Alrxa94h5 (e.g., altered splicing caused by a mutation in a splice donor or splice acceptor site);

8) non-wild type (i.e., abnormal) level of Alrxa94h5 protein in cells or extracellular material obtained from a subject;

9) loss of an allele of a gene encoding Alrxa94h5; and

10) inappropriate post-translational modification of a Alrxa94h5 protein.

3. Pharmacogenomics

Agents, or modulators which have a stimulatory or inhibitory effect on activity or expression of a polypeptide of the invention as identified by a screening assay described herein can be administered to individuals to treat (prophylactically or therapeutically) disorders associated with aberrant activity of the polypeptide. In conjunction with such treatment, the pharmacogenomics (i.e., the study of the relationship between an individual's genotype and that individual's response to a foreign compound or drug) of the individual can be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of a polypeptide of the invention, expression of a nucleic acid of the invention, or mutation content of a gene of the invention in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See, e.g., Linder (1997) Clin. Chem. 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism”. These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of a polypeptide of the invention, expression of a nucleic acid encoding the polypeptide, or mutation content of a gene encoding the polypeptide in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a modulator of activity or expression of the polypeptide, such as a modulator identified by one of the exemplary screening assays described herein.

Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a Alrxa94h5 molecule or Alrxa94h5 modulator as well as tailoring the dosage and/or therapeutic regimen of treatment with a Alrxa94h5 molecule or Alrxa94h5 modulator.

One pharmacogenomics approach (designated “genome-wide association”) to identifying genes that predict drug response relies on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared with a map of the genome of each of a statistically significant number of patients taking part in a phase II or phase III drug trial in order to identify markers associated with a particular drug response or side effect. Alternatively, such a high resolution map can be generated from a combination of many (e.g., millions or tens of millions) known single nucleotide polymorphisms (SNPs) in the human genome. As used herein, a SNP is an alteration of a single nucleotide residue that occurs at a particular position in a DNA sequence. For example, a SNP may occur once per every 1000 bases of DNA. A SNP may be involved in a disease process, however, the vast majority appear not to be disease-associated. Given a genetic map based on the occurrence of such SNPs, individuals can be grouped into genetic categories depending on a particular pattern of SNPs in their individual genome. In this manner, treatment regimens can be tailored to groups of genetically similar individuals, taking into account traits that may be common among such genetically similar individuals.

Alternatively, a method (designated a “candidate gene approach”) can be used to identify genes that are predictive of a certain drug response. According to this method, if a gene that encodes a target upon which a drug acts is known (e.g., a Alrxa94h5 protein), all common variants of that gene can be identified in the population and it can be determined whether having one version of the gene versus another is associated with a particular drug response.

As a further alternative, a method (designated “gene expression profiling”) can be used to identify genes that predict drug response. For example, identification of genes expressed in an animal to which a drug (e.g., a Alrxa94h5 molecule or Alrxa94h5 modulator of the present invention) has been administered can indicate whether gene pathways related to toxicity have been activated or enhanced.

Information generated from more than one of the above pharmacogenomics approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating a subject with a Alrxa94h5 molecule or Alrxa94h5 modulator, such as a modulator identified by one of the exemplary screening assays described herein.

The present invention further provides methods for identifying new agents, or combinations, that are based on identifying agents that modulate the activity of one or more of the gene products encoded by one or more of the Alrxa94h5 nucleic acids of the present invention, wherein these products may be associated with resistance of the cells to a therapeutic agent. Specifically, the activity of the proteins encoded by the Alrxa94h5 genes of the present invention can be used as a basis for identifying agents for overcoming agent resistance. By blocking the activity of one or more of the resistance proteins, target cells, e.g., bone cells, will become sensitive to treatment with an agent that the unmodified target cells were resistant to.

Other Embodiments

In another aspect, the invention features, a method of analyzing a plurality of capture probes. The method can be used, e.g., to analyze gene expression. The method includes: providing a two dimensional array having a plurality of addresses, each address of the plurality being positionally distinguishable from each other address of the plurality, and each address of the plurality having a unique capture probe, e.g., a nucleic acid or peptide sequence; contacting the array with a (preferably purified) Alrxa94h5 nucleic acid, polypeptide, or antibody, and thereafter evaluating the plurality of capture probes. Binding (e.g., in the case of a nucleic acid, hybridization with a capture probe at an address of the plurality) can detected, for example, by detection of a signal associated with a label attached to the Alrxa94h5 nucleic acid, polypeptide, or antibody.

The capture probes can be a set of nucleic acids from a selected sample, e.g., a sample of nucleic acids derived from a control or non-stimulated tissue or cell.

The method can include contacting the Alrxa94h5 nucleic acid, polypeptide, or antibody with a first array having a plurality of capture probes and a second array having a different plurality of capture probes. The results of each hybridization can be compared, e.g., to analyze differences in expression between a first and second sample. The first plurality of capture probes can be from a control sample, e.g., a wild type, normal, or non-diseased, non-stimulated, sample, e.g., a biological fluid, tissue, or cell sample. The second plurality of capture probes can be from an experimental sample, e.g., a mutant type, at risk, disease-state or disorder-state, or stimulated, sample, e.g., a biological fluid, tissue, or cell sample.

The plurality of capture probes can be a plurality of nucleic acid probes each of which specifically hybridizes, with an allele of Alrxa94h5. Such methods can be used to diagnose a subject, e.g., to evaluate risk for a disease or disorder, to evaluate suitability of a selected treatment for a subject, to evaluate whether a subject has a disease or disorder. Alrxa94h5 is associated with bone metabolism, thus it is useful for evaluating bone disorders.

The method can be used to detect SNPs, as described above.

In another aspect, the invention features, a method of analyzing a plurality of probes. The method is useful, e.g., for analyzing gene expression. The method includes: providing a two dimensional array having a plurality of addresses, each address of the plurality being positionally distinguishable from each other address of the plurality having a unique capture probe, e.g., wherein the capture probes are from a cell or subject which express Alrxa94h5 or from a cell or subject in which a Alrxa94h5 mediated response has been elicited, e.g., by contact of the cell with Alrxa94h5 nucleic acid or protein, or administration to the cell or subject Alrxa94h5 nucleic acid or protein; contacting the array with one or more inquiry probe, wherein an inquiry probe can be a nucleic acid, polypeptide, or antibody (which is preferably other than Alrxa94h5 nucleic acid, polypeptide, or antibody); providing a two dimensional array having a plurality of addresses, each address of the plurality being positionally distinguishable from each other address of the plurality, and each address of the plurality having a unique capture probe, e.g., wherein the capture probes are from a cell or subject which does not express Alrxa94h5 (or does not express as highly as in the case of the Alrxa94h5 positive plurality of capture probes) or from a cell or subject which in which a Alrxa94h5 mediated response has not been elicited (or has been elicited to a lesser extent than in the first sample); contacting the array with one or more inquiry probes (which is preferably other than a Alrxa94h5 nucleic acid, polypeptide, or antibody), and thereby evaluating the plurality of capture probes. Binding, e.g., in the case of a nucleic acid, hybridization with a capture probe at an address of the plurality, is detected, e.g., by signal generated from a label attached to the nucleic acid, polypeptide, or antibody.

In another aspect, the invention features, a method of analyzing Alrxa94h5, e.g., analyzing structure, function, or relatedness to other nucleic acid or amino acid sequences. The method includes: providing a Alrxa94h5 nucleic acid or amino acid sequence, e.g., a nucleotide sequence from 300-1916 or a portion thereof; comparing the Alrxa94h5 sequence with one or more preferably a plurality of sequences from a collection of sequences, e.g., a nucleic acid or protein sequence database; to thereby analyze Alrxa94h5.

The method can include evaluating the sequence identity between a Alrxa94h5 sequence and a database sequence. The method can be performed by accessing the database at a second site, e.g., over the internet.

In another aspect, the invention features, a set of oligonucleotides, useful, e.g., for identifying SNP's, or identifying specific alleles of Alrxa94h5. The set includes a plurality of oligonucleotides, each of which has a different nucleotide at an interrogation position, e.g., an SNP or the site of a mutation. In a preferred embodiment, the oligonucleotides of the plurality identical in sequence with one another (except for differences in length). The oligonucleotides can be provided with diferential labels, such that an oligonucleotides which hybridizes to one allele provides a signal that is distinguishable from an oligonucleotides which hybridizes to a second allele.

4. Monitoring of Effects During Clinical Trials

Monitoring the influence of agents (e.g., drugs compounds) on the expression or activity of a polypeptide of the invention (e.g., the ability to modulate aberrant cell proliferation chemotaxis, and/or differentiation) can be applied not only in basic drug screening, but also in clinical trials. For example, the effectiveness of an agent, as determined by a screening assay as described herein, to increase gene expression, protein levels, or protein activity, can be monitored in clinical trials of subjects exhibiting decreased gene expression, protein levels, or protein activity. Alternatively, the effectiveness of an agent, as determined by a screening assay, to decrease gene expression, protein levels or protein activity, can be monitored in clinical trials of subjects exhibiting increased gene expression, protein levels, or protein activity. In such clinical trials, expression or activity of a polypeptide of the invention and preferably, that of other polypeptide that have been implicated in for example, a cellular proliferation disorder, can be used as a marker of the immune responsiveness of a particular cell.

For example, and not by way of limitation, genes, including those of the invention, that are modulated in cells by treatment with an agent (e.g., compound, drug or small molecule) which modulates activity or expression of a polypeptide of the invention (e.g., as identified in a screening assay described herein) can be identified. Thus, to study the effect of agents on cellular proliferation disorders, for example, in a clinical trial, cells can be isolated and RNA prepared and analyzed for the levels of expression of a gene of the invention and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of a gene of the invention or other genes. In this way, the gene expression pattern can serve as a marker, indicative of the physiological response of the cells to the agent. Accordingly, this response state can be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of the polypeptide or nucleic acid of the invention in the pre-administration sample; (iii) obtaining one or more post-administration samples from the subject; (iv) detecting the level the of the polypeptide or nucleic acid of the invention in the post-administration samples; (v) comparing the level of the polypeptide or nucleic acid of the invention in the pre-administration sample with the level of the polypeptide or nucleic acid of the invention in the post-administration sample or samples; and (vi) altering the administration of the agent to the subject accordingly. For example, increased administration of the agent can be desirable to increase the expression or activity of the polypeptide to higher levels than detected, i.e., to increase the effectiveness of the agent. Alternatively, decreased administration of the agent can be desirable to decrease expression or activity of the polypeptide to lower levels than detected, i.e., to decrease the effectiveness of the agent.

C. Methods of Treatment

The present invention provides for both prophylactic and therapeutic methods of treating a subject at risk of (or susceptible to) a disorder or having a disorder associated with aberrant expression or activity of a polypeptide of the invention and/or in which the polypeptide of the invention is involved. For example, disorders characterized by aberrant expression or activity of the polypeptides of the invention include disorders of nerve, neuronal, and muscle cells, as well as disorders related to aberrant nerve or skeletal muscle impulse transmission. Exemplary disorders include epilepsy, depression, mood disorders, and pain, as well as other uses described herein.

1. Prophylactic Methods

In one aspect, the invention provides a method for preventing in a subject, a disease or condition associated with an aberrant expression or activity of a polypeptide of the invention, by administering to the subject an agent which modulates expression or at least one activity of the polypeptide. Subjects at risk for a disease which is caused or contributed to by aberrant expression or activity of a polypeptide of the invention can be identified by, for example, any or a combination of diagnostic or prognostic assays as described herein. Administration of a prophylactic agent can occur prior to the manifestation of symptoms characteristic of the aberrance, such that a disease or disorder is prevented or, alternatively, delayed in its progression. Depending on the type of aberrance, for example, an agonist or antagonist agent can be used for treating the subject. The appropriate agent can be determined based on screening assays described herein.

2. Therapeutic Methods

Another aspect of the invention pertains to methods of modulating expression or activity of a polypeptide of the invention for therapeutic purposes. The modulatory method of the invention involves contacting a cell with an agent that modulates one or more of the activities of the polypeptide. An agent that modulates activity can be an agent as described herein, such as a nucleic acid or a protein, a naturally-occurring cognate ligand of the polypeptide, a peptide, a peptidomimetic, or other small molecule. In one embodiment, the agent stimulates one or more of the biological activities of the polypeptide. Examples of such stimulatory agents include the active polypeptide of the invention and a nucleic acid molecule encoding the polypeptide of the invention that has been introduced into the cell. In another embodiment, the agent inhibits one or more of the biological activities of the polypeptide of the invention. Examples of such inhibitory agents include antisense nucleic acid molecules and antibodies. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a polypeptide of the invention. In one embodiment, the method involves administering an agent (e.g., an agent identified by a screening assay described herein), or combination of agents that modulates (e.g., up-regulates or down-regulates) expression or activity. In another embodiment, the method involves administering a polypeptide of the invention or a nucleic acid molecule of the invention as therapy to compensate for reduced or aberrant expression or activity of the polypeptide.

Stimulation of activity is desirable in situations in which activity or expression is abnormally low or down-regulated and/or in which increased activity is likely to have a beneficial effect, e.g., in wound healing. Conversely, inhibition of activity is desirable in situations in which activity or expression is abnormally high or up-regulated and/or in which decreased activity is likely to have a beneficial effect.

### EXAMPLES

The invention is now described with reference to the following Examples. These Examples are provided for the purpose of illustration only and the invention is not limited to these Examples, but rather encompasses all variations which are evident as a result of the teaching provided herein.

The β3 sodium channel subunit described herein is a newly discovered β subunit which exhibits significant sequence homology with the β1 subunit. In order to examine the functional consequences of β subunit co-expression, tsA-201 cells were co-transformed with rat brain type IIA sodium channel a subunits alone, with one of the β subunits, or with combinations (e.g. β1+β2 or β2+β33) of subunits. Transformation of cells with plasmids encoding a single β subunit, pairs of β subunits, or with the α subunit caused depolarizing shifts in the voltage dependence of activation and inactivation. The largest shifts (i.e. about +12 millivolts) occurred in cells which were transformed with β3 or with β2+β3.

Co-transformation of cells with the a subunit and with β1, β2, or β1+β2 did not change the rate of sodium current inactivation during pulses to positive potentials, and there was little non-inactivating current. In contrast, co-transformation of cells with the α subunit and with β3 or β2+β3 slowed inactivation of current during depolarization, and inactivation was less complete (i.e. 4.5% versus 1.2 sustained current).

Presence of β3 sodium channel subunit in a cell favors sustained, non-inactivating sodium current, including sodium currents having important neurophysiological roles in, for example, nociception.

Electrophysiology Experiments in Transformed Cells

Transient expression of one or more sodium channel subunits was achieved by co-transforming tsA-201 cells (IEK 293 cells which express large T-antigen; obtainable from CellGenesys, Foster City, Calif.) with one or more plasmid vectors encoding individual sodium channel subunits (i.e. one or more of plasmid pCDM8α, encoding the α subunit; plasmid pCDM8β1, encoding the β1 subunit; plasmid pCDM8β2, encoding the β2 subunit; and plasmid pMET7, encoding the β3 subunit). A standard calcium phosphate precipitation method was used for transformation. The cells were also co-transformed with EBO-CDI, i.e. a plasmid vector designated EBO comprising an insert encoding CD8, in order to facilitate identification of transformed cells, as described (Jurman et al., 1994, BioTechniques 17:876-881).

Transformed cells were sub-cultured on the day following transformation and analyzed in the period 40-52 hours following transformation. Cells which expressed the CD8 antigen (i.e. transformed cells) were identified by incubating cells with polystyrene microspheres that had been pre-coated with antibody which binds specifically with CD8. Cells which bound with the microspheres were isolated and used for electrophysiological experiments.

Whole-cell voltage clamp experiments were performed according to standard methods, using solution A in the pipette and solution B in the bath. Solution A comprised 189 millimolar N-methyl D-glucamine (NMDG), 1 millimolar NaCl, 4 millimolar MgCl₂, 0.1 millimolar BAPTA, 25 millimolar phosphocreatine (Tris salt), 2 millimolar ATP (sodium salt), 0.2 millimolar GTP (sodium salt), and 40 millimolar HEPES, wherein the pH was adjusted to 7.2 using H₂SO₃. Solution B comprised 140 millimolar NaCl, 5.4 millimolar CsCl, 1.8 millimolar CaCl₂, 1 millimolar MgCl₂, and 10 millimolar HEPES, wherein the pH was adjusted to 7.4.

Determination of Mean Voltage Dependence of Activation and Inactivation

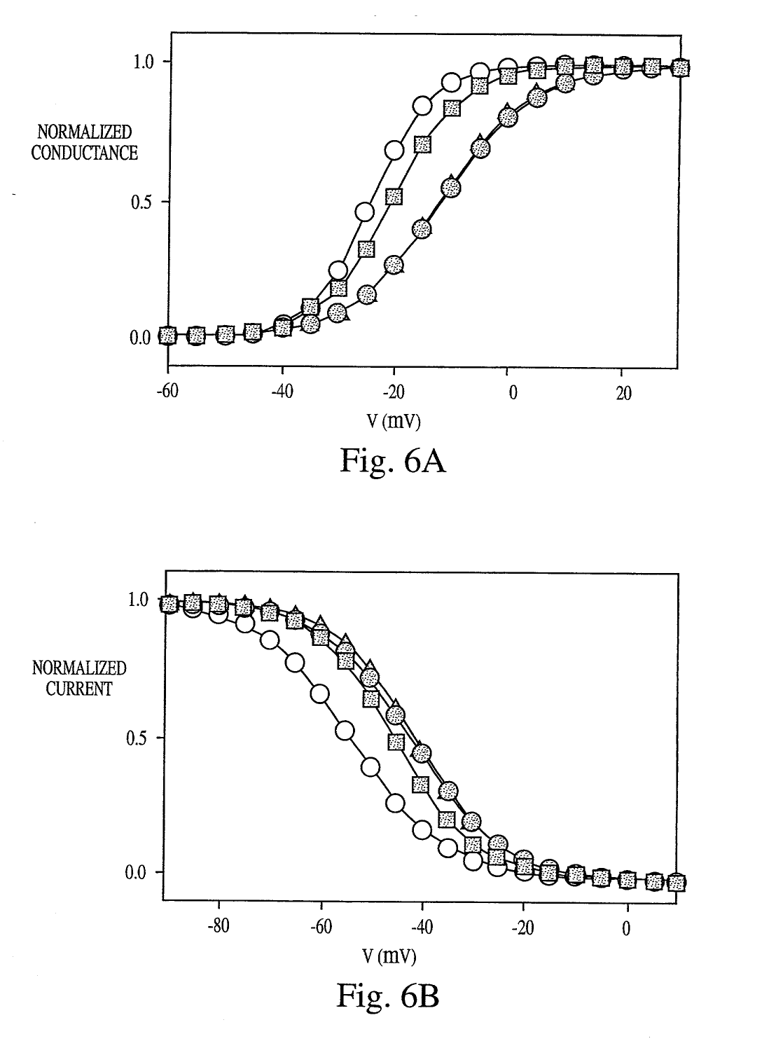

Mean voltage dependence of activation was determined using current-voltage relationships assessed using individual transformed cells. These conductance-voltage relationships (examples shown in FIG. 6A) was fitted to the Boltzmann equation in order to determine values for the half activation voltage (V_{½}) and a slope factor (k). The following values of V_{½} and k were determined for the indicated transformed cells:

among 25 cells transformed with a subunit alone, the average value of V_{½} was −24.3±0.8 millivolts (mV), and the average value of k was −5.3±0.1 mV;

among 12 cells transformed with α and β2 subunits, the average value of V_{½}was −20.7±0.7 mV, and the average value of k was −6.4±0.2 mV;

among 15 cells transformed with α and β3 subunits, the average value of V_{½} was ±12.1±2.0 mV, and the average value of k was ±8.3±0.6 mV; and

among 19 cells transformed with α, β2, and β3 subunits, the average value of V_{½} was ±12.1±2.0 mV, and the average value of k was −8.3±0.6 mV.

Mean voltage dependence of inactivation was determined using current-voltage relationships assessed using individual transformed cells. In these experiments, the cells were conditioned by applying a 20 millisecond pre-pulse. A test pulse to 0 millivolts was then applied, from a holding potential of −100 millivolts. Peak test pulse current values obtained from individual experiments at pre-pulse voltage (V′) were plotted as a function of pre-pulse potential and fit to the Boltzmann equation in order to determine values for the half inactivation voltage (V′_{½}) and a slope factor (k). The following values of V′_{½} and k were determined for the indicated transformed cells:

among 20 cells transformed with a subunit alone, the average value of V′_{½} was −53.4±1.5 millivolts (mV), and the average value of k was 8.9+0.2 mV;

among 12 cells transformed with α and β2 subunits, the average value of V′_{½} was −44.9±2.0 mV, and the average value of k was 7.6±0.4 mV; and

among 12 cells transformed with α and β3 subunits, the average value of V′_{½} was −41.5+2.7 mV, and the average value of k was 8.6±0.7 mV; and

among 14 cells transformed with α, β2, and β3 subunits, the average value of V′_{½} was −40.9±1.8 mV, and the average value of k was 7.8±0.6 mV.

These data demonstrate that co-expression of sodium channel α and β3 subunits shifts the voltage dependence of activation and inactivation of the channel toward more positive values (i.e. causes these values to become less negative).

Recordation of Transient Sodium Currents

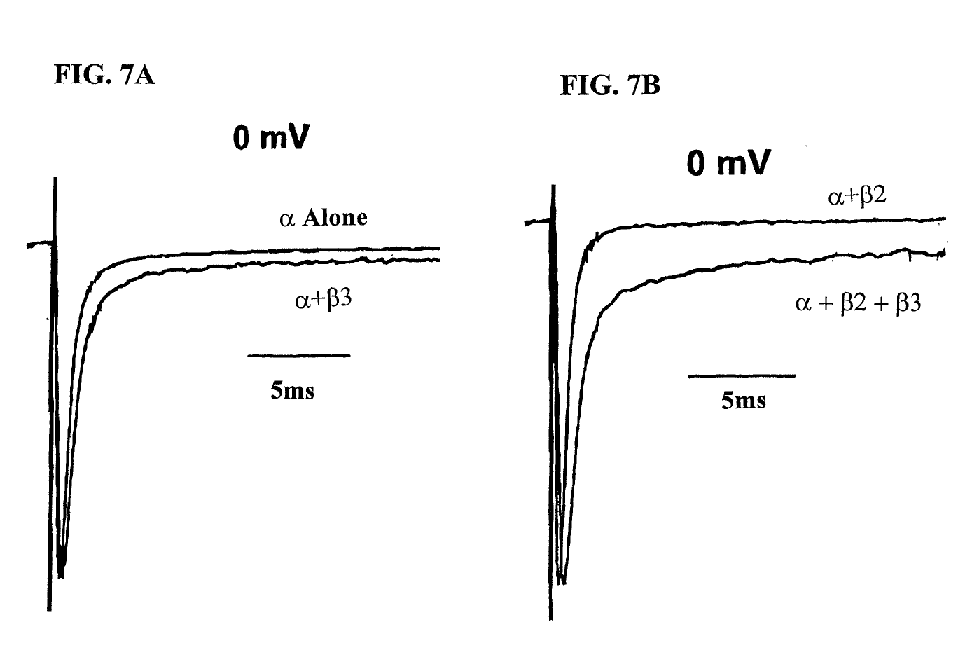

Transient sodium currents were elicited in tsA-201 cells transformed with one or more plasmid vectors encoding individual sodium channel subunits, as described above. Currents were elicited by applying test pulses to 0, 20, or 30 millivolts (from a holding potential of −90 millivolts) to individual cells. Representative electrographic tracings obtained using the electrode procedure described above are shown in FIG. 7. From 10 to 25 individual current recordings were used to generate normalized current records for each type of transformed cell. It was found that cells which were not transformed with a plasmid encoding the β3 subunit exhibited a faster and more completely inactivating current than cells transformed with the same sodium channel subunit(s) and also with the β3 subunit. Co-expression of β3 subunit did not change the recovery from inactivation at −90 millivolts. These data also demonstrate that the β3 subunit of sodium channel proteins modulates the voltage-dependence of activation and inactivation of the channel.

All publications, patents, and patent applications referenced in this specification are incorporated herein by reference to the same extent as if each individual publication, patent, or patent application had been specifically and individually indicated to be incorporated herein by reference.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. An isolated nucleic acid molecule selected from the group consisting of:
a) a nucleic acid molecule having a nucleotide sequence which is at least 40% identical to the nucleotide sequence of SEQ ID NO: 1, 21, 22, or a complement thereof;
b) a nucleic acid molecule comprising at least 437 nucleotide residues and having a nucleotide sequence identical to at least 437 consecutive nucleotide residues of SEQ ID NO: 1, 21, 22, or a complement thereof;
c) a nucleic acid molecule comprising at least 15 nucleotide residues and having a nucleotide sequence identical to at least 15 consecutive nucleotide residues of SEQ ID NO: 21 or 22, or a complement thereof;
d) a nucleic acid molecule which encodes a polypeptide comprising the amino acid sequence of SEQ ID NO: 2 or 4;
e) a nucleic acid molecule which encodes a fragment of a polypeptide comprising the amino acid sequence of SEQ ID NO: 2 or 4, wherein the fragment comprises at least 8 consecutive amino acid residues of SEQ ID NO: 2 or 4; and
f) a nucleic acid molecule which encodes a naturally occurring allelic variant of a polypeptide comprising the amino acid sequence of SEQ ID NO: 2 or 4, wherein the nucleic acid molecule hybridizes to a nucleic acid molecule consisting of the nucleotide sequence of SEQ ID NO: 1, 21, 22, or a complement thereof under stringent conditions.

2. The isolated nucleic acid molecule of claim 1, which is selected from the group consisting of:
a) a nucleic acid having the nucleotide sequence of SEQ ID NO: 1, 21, 22, or a complement thereof; and
b) a nucleic acid molecule which encodes a polypeptide having the amino acid sequence of SEQ ID NO: 2 or 12.

3. The nucleic acid molecule of claim 1, further comprising vector nucleic acid sequences.

4. The nucleic acid molecule of claim 1 further comprising nucleic acid sequences encoding a heterologous polypeptide.

5. A host cell which contains the nucleic acid molecule of claim 1.

6. The host cell of claim 5 which is a mammalian host cell.

7. A non-human mammalian host cell containing the nucleic acid molecule of claim 1.

8. An isolated polypeptide selected from the group consisting of:
a) a fragment of a polypeptide comprising the amino acid sequence of SEQ ID NO: 2 or 4, wherein the fragment comprises at least 8 contiguous amino acids of SEQ ID NO: 2 or 4;
b) a naturally occurring allelic variant of a polypeptide comprising the amino acid sequence of SEQ ID NO: 2 or 4, wherein the polypeptide is encoded by a nucleic acid molecule which hybridizes to a nucleic acid molecule consisting of the nucleotide sequence of SEQ ID NO: 1, 21, 22, or a complement thereof under stringent conditions; and
c) a polypeptide which is encoded by a nucleic acid molecule comprising a nucleotide sequence which is at least 40% identical to a nucleic acid consisting of the nucleotide sequence of SEQ ID NO: 2, 21, 22, or a complement thereof.

9. The isolated polypeptide of claim 8 having the amino acid sequence of SEQ ID NO: 2 or 4.

10. The polypeptide of claim 8, wherein the amino acid sequence of the polypeptide further comprises heterologous amino acid residues.

11. An antibody which selectively binds with the polypeptide of claim 8.

12. A method for producing a polypeptide selected from the group consisting of:
a) a polypeptide comprising the amino acid sequence of SEQ ID NO: 2 or 4;
b) a polypeptide comprising a fragment of the amino acid sequence of SEQ ID NO: 2 or 4, wherein the fragment comprises at least 8 contiguous amino acids of SEQ ID NO: 2 or 4; and
c) a naturally occurring allelic variant of a polypeptide comprising the amino acid sequence of SEQ ID NO: 2 or 4, wherein the polypeptide is encoded by a nucleic acid molecule which hybridizes to a nucleic acid molecule consisting of the nucleotide sequence of SEQ ID NO: 1, 21, 22, or a complement thereof under stringent conditions;
the method comprising culturing the host cell of claim 5 under conditions in which the nucleic acid molecule is expressed.

13. A method for detecting the presence of a polypeptide of claim 8 in a sample, comprising:
a) contacting the sample with a compound which selectively binds with a polypeptide of claim 8; and
b) determining whether the compound binds with the polypeptide in the sample.

14. The method of claim 13, wherein the compound which binds with the polypeptide is an antibody.

15. A kit comprising a compound which selectively binds with a polypeptide of claim 8 and instructions for use.

16. A method for detecting the presence of a nucleic acid molecule of claim 1 in a sample, comprising the steps of:
a) contacting the sample with a nucleic acid probe or primer which selectively hybridizes with the nucleic acid molecule; and
b) determining whether the nucleic acid probe or primer binds with a nucleic acid molecule in the sample.

17. The method of claim 16, wherein the sample comprises mRNA molecules and is contacted with a nucleic acid probe.

18. A kit comprising a compound which selectively hybridizes with a nucleic acid molecule of claim 1 and instructions for use.

19. A method for identifying a compound which binds with a polypeptide of claim 8, the method comprising the steps of:
a) contacting a polypeptide, or a cell expressing a polypeptide of claim 8 with a test compound; and
b) determining whether the polypeptide binds with the test compound.

20. The method of claim 19, wherein the binding of the test compound with the polypeptide is detected by a method selected from the group consisting of:
a) detection of binding by direct detecting of test compound/polypeptide binding;
b) detection of binding using a competition binding assay;
c) detection of binding using an assay for sodium ion transmembrane transport.

21. A method for modulating the activity of a polypeptide of claim 8 comprising contacting the polypeptide or a cell expressing the polypeptide of claim 8 with a compound which binds with the polypeptide in a sufficient concentration to modulate the activity of the polypeptide.

22. A method for identifying a compound which modulates the activity of a polypeptide of claim 8, comprising:
a) contacting the polypeptide of claim 8 with a test compound; and
b) determining the effect of the test compound on the activity of the polypeptide to thereby identify a compound which modulates the activity of the polypeptide.

23. An antibody substance which selectively binds to the polypeptide of claim 8, wherein the antibody substance is made by providing the polypeptide to an immunocompetent vertebrate and thereafter harvesting blood or serum from the vertebrate.
